(19) United States
(12) Patent Application Publication
(10) Pub. No.: US 2026/0096771 A1
(43) Pub. Date: Apr. 9, 2026

(54) Multi-Layer Contact Patch to Perform an Allergen Diagnostic

(71) Applicant: Evme Inc., Los Altos, CA (US)

(72) Inventors: Anjuli Kumar MEHROTRA, Los Altos, CA (US); Yun-Ling WONG, Seattle, WA (US)

(21) Appl. No.: 18/927,033

(22) Filed: Oct. 25, 2024

Related U.S. Application Data
(63) Continuation of application No. 17/882,110, filed on Aug. 5, 2022.
Application No. 17/882,110 is a continuation of application No. PCT/US2021/059924, filed on Nov. 18, 2021.
(60) Provisional application No. 63/115,417, filed on Nov. 18, 2020.

(51) Int. Cl. A61B 5/00; A61B 10/00; G16H 15/00; G16H 80/00

(52) U.S. Cl. CPC A61B 5/411; A61B 5/0013; A61B 5/0077; A61B 5/6832; A61B 5/7475; A61B 10/0035; G16H 80/00; G16H 15/00

(57) ABSTRACT

Multi-layer contact patches, as well as related apparatus, kits, systems, and methods, enable improved allergen testing and diagnosis. Multi-layer contact patches include a shield layer releasably coupled to an adhesive layer. The adhesive layer at least partially defines and is fixably coupled to an allergen complex layer. The allergen complex layer defines a plurality of allergen complex cavities. At least a subset of the allergen complex cavities include an allergen complex positioned therein, each allergen complex comprising an absorbent matrix and an allergen.

### CROSS-REFERENCE TO RELATED APPLICATIONS

This application is a continuation of U.S. patent application Ser. No. 17/882,110, filed Aug. 5, 2022, entitled “APPARATUS TO PERFORM AN ALLERGEN DIAGNOSTIC” (Attorney Docket No. PACH-0001-U01).

U.S. patent application Ser. No. 17/882,110 is a continuation of International Application No. PCT/US21/59924, filed Nov. 18, 2021, entitled “SYSTEM, METHOD, AND APPARATUS TO PERFORM AN ALLERGEN DIAGNOSTIC” (Attorney Docket No. PACH-0001-WO).

PCT/US21/59924, filed Nov. 18, 2021 (Attorney Docket No. PACH-0001-WO) claims priority to U.S. Provisional Patent Application No. 63/115,417, filed on Nov. 18, 2020 entitled “SYSTEM, METHOD, AND APPARATUS TO PERFORM AN ALLERGEN DIAGNOSTIC” (Attorney Docket No. PACH-0001-P01).

Each of the foregoing applications is incorporated herein by reference in their entireties for all purposes.

### BACKGROUND

Previously known allergen patch systems suffer from a number of drawbacks. Allergen panels are limited to a number of pre-determined allergens for the test. Execution of previously known allergen panel tests further requires close monitoring and execution by a medical provider, which may include access to a specialist. Accordingly, patients in many locations may not have practical access to a quality allergen panel test. Additionally, previously known allergen patch systems are difficult to execute properly, and are subject to significant poor sensitivity values in performing the test, and reduced confidence in conclusions based on the test.

### SUMMARY

An example system includes a multi-layer contact patch including a shield layer releasably coupled to an adhesive layer; the adhesive layer at least partially defining and fixably coupled to an allergen complex layer; the allergen complex layer defining a plurality of allergen complex cavities; and wherein at least a subset of the plurality of allergen complex cavities comprise an allergen complex positioned therein, each allergen complex comprising an absorbent matrix and an allergen.

Certain further aspects of the example system are described following, any one or more of which may be present in certain embodiments. The example system further includes a sealing layer interposed between the shield layer and the plurality of allergen complex cavities, wherein the sealing layer is releasably coupled to the shield layer and the plurality of allergen complex cavities. The example system further includes wherein the allergen complex layer defines between 2 and 48 allergen complex cavities, inclusive. The example system further includes wherein the allergen complex layer defines 16 allergen complex cavities. The example system further includes wherein the adhesive layer further comprises a perforation pattern. The example system further includes wherein the perforation pattern comprises a plurality of perforations between adjacent ones of the plurality of allergen complex cavities. The example system further includes wherein the perforation pattern comprises a plurality of perforations between adjacent groups of the plurality of allergen complex cavities. The example system further includes wherein the allergen complex layer further comprises a scoring pattern. The example system further includes wherein the plurality of allergen complex cavities comprise a shape pattern comprising at least two distinct shapes, wherein each of the plurality of allergen complex cavities comprise a selected one of the at least two distinct shapes. The example system further includes at least one orientation feature, each of the at least one orientation features comprising an opening extending through at least one of the adhesive layer or the allergen complex layer. The example system further includes at least one alignment feature, each of the at least one alignment features comprising an opening extending through at least one of the adhesive layer or the allergen complex layer. The example system further includes wherein each one of the allergen complexes comprises a distinct allergen composition. The example system further includes wherein the distinct allergen compositions of the allergen complexes are selected to form an allergen panel. The example system further includes wherein the allergen panel comprises a selected group of allergens according to an expected population incidence rate. The example system further includes wherein the allergen panel comprises a selected group of allergens according to an exposure profile. The example system further includes wherein the exposure profile comprises an industry specific exposure profile. The example system further includes wherein the allergen panel comprises at least one of a mining panel, an agriculture panel, a metal panel, a pediatric panel, a fragrance panel, an oil and gas panel, a refining panel, a manufacturing panel, a carpentry panel, a health care panel, a hospital panel, a dye panel, a textile panel, surgical panel, a dental panel, a hairdresser panel, a specified exposure panel, and/or a food panel. The example system further includes wherein at least one of the allergen complexes comprises a control composition.

An example apparatus includes a user introduction circuit structured to provide an allergen patch execution instruction set to a user device; an allergen test execution circuit structured to: determine a patch removal event; capture a first test area image in response to the patch removal event; provide a user notification in response to an elapsed time value determined from the patch removal event; and capture a second test area image in response to the elapsed time value; and a provider interaction circuit structured to transmit the first test area image and the second test area image to a provider device.

Certain further aspects of the example apparatus are described following, any one or more of which may be present in certain embodiments. The example apparatus further includes wherein the allergen patch execution instruction set comprises a patch placement instruction. The example apparatus further includes wherein the user introduction circuit is further structured to provide a test exclusion criteria to the user device, and to determine a test approval value in response to a user response to the test exclusion criteria. The example apparatus further includes wherein the allergen test execution circuit is further structured to: photonically interrogate a test area; to determine a test execution approval in response to the photonically interrogating; and to provide the test execution approval to the user device. The example apparatus further includes wherein the allergen test execution circuit is further structured to determine the test execution approval in response to at least one of a test area geometry value or a test area clear value. The example apparatus further includes wherein the provider interaction circuit is further structured to provide a third test area image to the provider device in response to the photonically interrogating. The example apparatus further includes wherein the provider interaction circuit is further structured to provide the test execution approval to the provider device. The example apparatus further includes wherein the allergen patch execution instruction set further comprises a test execution schedule. The example apparatus further includes wherein the allergen test execution circuit is further structured to perform at least one of a de-skew operation, an orientation operation, and/or a scaling operation to at least one of the first test area image or the second test area image. The example apparatus further includes wherein the allergen test execution circuit is further structured to interpret at least one of an alignment feature or an orientation feature, and wherein the operation to perform the at least one of the de-skew operation, the orientation operation, and/or the scaling operation is performed in response to the at least one of the alignment feature or the orientation feature. The example apparatus further includes wherein the allergen test execution circuit is further structured to provide a user assistance overlay, and to capture the first test area image after providing the user assistance overlay. The example apparatus further includes wherein the allergen test execution circuit is further structured to: photonically interrogate a test area; determine that an acceptable image for the first test area image is available in response to the photonically interrogating; and in response to determining the acceptable image for the first test area image is available, capture the first test area image and provide a user feedback value. The example apparatus further includes wherein the allergen test execution circuit is further structured to: photonically interrogate a test area; determine that the user device is acceptably positioned for capturing the first test area image in response to the photonically interrogating; and in response to determining the user device is acceptably positioned, capture the first test area image and provide a user feedback value. The example apparatus further includes wherein the allergen test execution circuit is further structured to capture a first test area video in response to the patch removal event. The example apparatus further includes wherein the first test area image comprises a selected image from the first test area video. The example apparatus further includes wherein the provider interaction circuit is further structured to transmit the first test area video to the provider device. The example apparatus further includes wherein the provider interaction circuit is further structured to interpret an allergen scoring value from the provider device; wherein the allergen test execution circuit is further structured to generate an allergen report in response to the allergen scoring value; and a user interaction circuit structured to provide the allergen report to the user device. The example apparatus further includes wherein the allergen report includes at least one of: an allergen ingredient value; an allergen chemical description value; and/or an offset allergen description value. The example apparatus further includes wherein the user interaction circuit is further structured to interpret a candidate product description value; wherein the allergen test execution circuit is further structured to determine a product compatibility value in response to the allergen report and the candidate product description value; and wherein the user interaction circuit is further structured to provide the product compatibility value to the user device. The example apparatus further includes wherein the allergen test execution circuit is further structured to determine a medical follow-up description in response to the allergen report; and wherein the user interaction circuit is further structured to provide the medical follow-up description to the user device. The example apparatus further includes wherein the user interaction circuit is further structured to provide a follow-up notification value to the user device in response to the medical follow-up description. The example apparatus further includes wherein the medical follow-up description comprises at least one of: a follow-up test value; a repeat test value; an allergen panel description value; a treatment plan description value; and/or a product description value. The example apparatus further includes wherein the provider interaction circuit is further structured to provide the allergen report to the provider device, and to interpret a provider command value from the provider device; and wherein the allergen test execution circuit is further structured to determine the medical follow-up description in response to the provider command value. The example apparatus further includes wherein the medical follow-up description comprises at least one of: a follow-up test value; a repeat test value; an allergen panel description value; a treatment plan description value; a treatment initiation value; and/or a product description value. The example apparatus further includes a test initiation circuit structured to operate a video conferencing interface between the user device and a support personnel device. The example apparatus further includes wherein the test initiation circuit is further structured to provide at least a portion of the allergen patch execution instruction set to at least one of the user device or the support personnel device during the operating the video conferencing interface.

An example apparatus includes an allergen patch comprising: a shield layer releasably coupled to an adhesive layer; the adhesive layer at least partially defining and fixably coupled to an allergen complex layer; the allergen complex layer defining a plurality of allergen complex cavities, and an allergen panel comprising a plurality of allergen complexes each positioned within at least one of the plurality of allergen complex cavities; and an instruction description comprising at least one of an allergen patch application instruction or an allergen application access instruction.

Certain further aspects of the example apparatus are described following, any one or more of which may be present in certain embodiments. The example apparatus further includes wherein the allergen patch further comprises an orientation feature. The example apparatus further includes wherein the allergen patch further comprises an alignment feature. The example apparatus further includes a hypo-allergenic marker. The example apparatus further includes a mobile phone screen template. The example apparatus further includes wherein the allergen patch further comprises a sealing layer interposed between the shield layer and the allergen complex layer. The example apparatus further includes a treatment overlay. The example apparatus further includes wherein the treatment overlay comprises a perforation pattern. The example apparatus further includes wherein the treatment overlay comprises a scoring pattern.

An example method includes removing a shield layer from a multi-layer contact patch; removing a sealing layer from the multi-layer contact patch; and applying an adhesive layer of the multi-layer contact patch to a skin test region, thereby contacting an allergen complex layer to the skin test region.

Certain further aspects of the example method are described following, any one or more of which may be present in certain embodiments. The example method further includes removing the adhesive layer from the skin test region; capturing and transmitting a first image of the skin test region; and capturing and transmitting a second image of the skin test region in response to an expiration of an elapsed time period after the removing the adhesive layer. The example method further includes wherein the capturing and transmitting the first image comprises utilizing a mobile phone screen template. The example method further includes wherein the capturing and transmitting the second image comprises utilizing the mobile phone screen template. The example method further includes marking the skin test region at an alignment feature of the multi-layer contact patch. The example method further includes marking the skin test region at an orientation feature of the multi-layer contact patch. The example method further includes wherein applying the adhesive layer further comprises applying an alignment marking to the skin test region. The example method further includes wherein applying the adhesive layer further comprises applying an orientation marking to the skin test region.

An example apparatus includes an allergen test execution circuit structured to: determine a patch removal event of an allergen patch; capture a first test area image in response to the patch removal event; provide a user notification in response to an elapsed time value determined from the patch removal event; and capture a second test area image in response to the elapsed time value; and a provider interaction circuit structured to transmit the first test area image and the second test area image to a provider device.

Certain further aspects of the example apparatus are described following, any one or more of which may be present in certain embodiments. The example apparatus further includes an allergen test evaluation circuit structured to: determine an allergen test success value in response to at least one of the patch removal event, the first test area image, and/or the second test area image; and perform a test execution adjustment in response to the allergen test success value. The example apparatus further includes wherein the allergen test success value comprises at least one value selected from the values consisting of: a first test area image description; a second test area image description; a patch placement description; a patch orientation description; a predicted allergen response description; and/or a predicted patient response trajectory. The example apparatus further includes wherein the patch removal event, the first test area image, and the second test area image correspond to a first test event; and wherein the allergen test evaluation circuit is further structured to determine a plurality of allergen test success values each corresponding to at least one additional test event, and to perform the test execution adjustment further in response to the at least one additional test event. The example apparatus further includes wherein the allergen test success value comprises at least one value selected from the values consisting of: a predicted allergen prevalence value; a predicted trajectory of allergen prevalence values; and/or a patient attribute correlation. The example apparatus further includes wherein the test execution adjustment comprises at least one adjustment selected from the adjustments consisting of: adjusting an allergen panel; adjusting an allergen concentration value; adjusting an allergen matrix composition; adjusting at least one of an alignment feature or an orientation feature of a subsequent allergen patch; adjusting the elapsed time value; adjusting at least one of a de-skew operation, an alignment operation, or a scaling operation; adjusting an image lighting parameter; adjusting an acceptable image determination value; adjusting a user feedback value provided in response to determining whether an acceptable image is provided and/or determining whether a user device is acceptably positioned for capturing the first test area image and/or the second test area image; adjusting a user assistance overlay utilized to assist a user in capturing the first test area image and/or the second test area image; adjusting a number of images to be captured for the first test area image and/or the second test area image; adjusting a user device position value for capturing the first test area image and/or the second test area image; adjusting at least one of an allergen patch application instruction or an allergen application access instruction; and/or adjusting at least one of an arrangement, spacing, and/or shape of allergen contact regions of a subsequent allergen patch. The example apparatus further includes wherein the provider interaction circuit is further structured to interpret an allergen test feedback value from the provider device; and wherein determining the allergen test success value is performed in response to the allergen test feedback value. The example apparatus further includes wherein the allergen test evaluation circuit is further structured to train on a corpus of data including a plurality of allergen test outcome values. The example apparatus further includes wherein the allergen test evaluation circuit is further structured to operate a neural network to perform at least one of determining the allergen test success value or the test execution adjustment. The example apparatus further includes wherein the allergen test evaluation circuit is further structured to operate an expert system to perform at least one of determining the allergen test success value or the test execution adjustment. The example apparatus further includes wherein the allergen test evaluation circuit is further structured to iteratively improve a trajectory of allergen test success values. The example apparatus further includes wherein the allergen test evaluation circuit is further structured to iteratively improve a trajectory of allergen test success values.

### BRIEF DESCRIPTION OF THE FIGURES

FIG. 1 is a schematic side view depiction of an allergen patch.

FIG. 2 is a schematic depiction of an allergen patch.

FIG. 3 is a schematic depiction of a system for performing an allergen patch test.

FIG. 4 is a schematic depiction of an overlay and/or a patch.

FIG. 5 is a schematic side view depiction of an allergen patch.

FIG. 6 is a schematic side view depiction of an allergen patch having a shielding layer.

FIG. 7 is a schematic depiction of an overlay and/or a patch.

FIG. 8 is a schematic depiction of an overlay and/or a patch.

FIG. 9 is a schematic depiction of an overlay and/or a patch.

FIG. 10 is a schematic depiction of an overlay and/or a patch.

FIG. 11 is a schematic diagram of an apparatus for performing an allergen patch test.

FIG. 12 is a schematic depiction of an allergen patch kit.

FIG. 13 is a schematic diagram of an apparatus for performing and iteratively improving an allergen patch test.

FIG. 14 is a schematic depiction of an overlay and/or a patch.

FIG. 15 is a schematic depiction of an overlay and/or a patch.

FIG. 16 is a schematic flow diagram of a procedure for providing test information of an allergen test to a medical provider.

FIG. 17 is a schematic flow diagram of a procedure to capture and/or enhance a test area image.

FIG. 18 is a schematic flow diagram of a procedure for applying an allergen patch.

FIG. 19 is a schematic flow diagram of a procedure to perform an allergen test.

FIG. 20 is a schematic flow diagram of a procedure for evaluating and improving an allergen test execution operation.

FIG. 21 is a schematic flow diagram of a procedure for iteratively improving a trajectory of allergen test success values.

### DETAILED DESCRIPTION

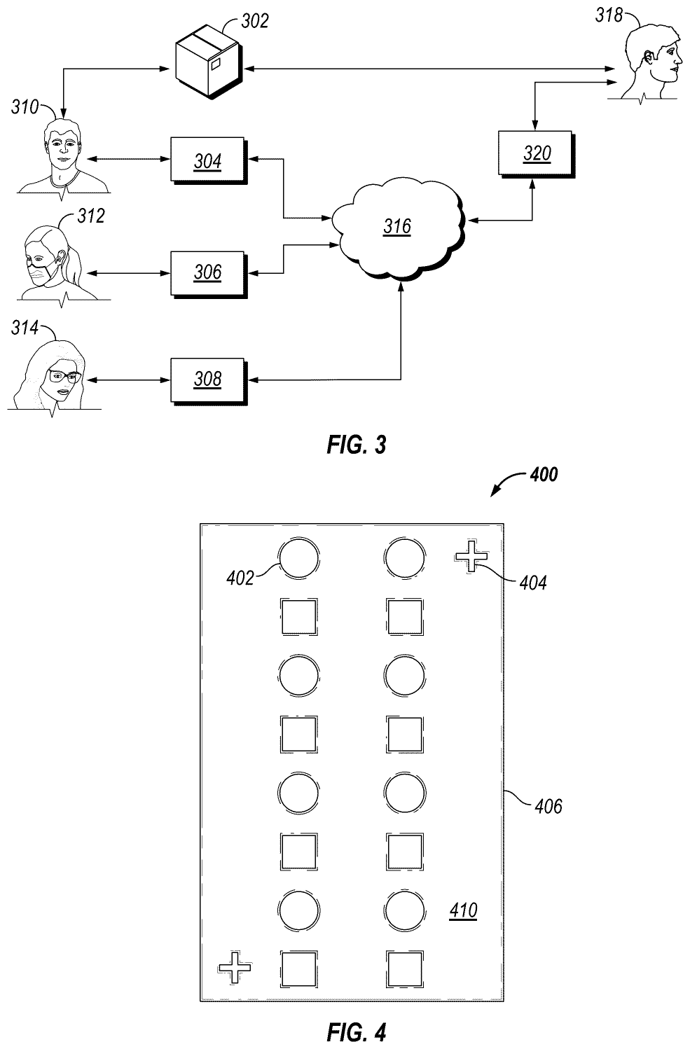

Referencing FIG. 3, an example system is depicted for providing, performing, and evaluating an allergen panel test. The example system references a user 310 (e.g., a patient, or the target person for which the allergen panel test is to be executed), a medical provider 312 (e.g., a doctor, physician's assistant, nurse practitioner, or other medical provider) performing and/or utilizing a result of the allergen panel test to treat and/or diagnose the user, and a support personnel 314 (e.g., a technician, nurse, and/or customer service representative), performing any operations described herein in relation to a support person. An example system further includes an allergen test provider 318, for example a person or entity providing the allergen patch (e.g., allergen patch kit 302) to be utilized in the allergen test panel, and/or providing for supporting operations for executing the test as set forth throughout the present disclosure. It will be understood that, while individuals are depicted in the various roles (user, medical provider, support personnel, and/or allergen test provider) for clarity of the present description, one or more of the depicted individuals may be an entity or group (e.g., a company, contractor, medical practice group, insurance company, etc.), and/or a given entity or group may provide all or a portion of operations described in relation to more than one role. For example, a medical provider 312 may perform operations related to the medical provider role, as well as operations related to the allergen test provider and/or support personnel role. In another example, a support person 314 and/or allergen test provider 318 may be a contractor that performs some operations related to the allergen test provider role and the support personnel role, and a medical provider 312 may perform other operations related to the allergen test provider role and the support personnel role. In another example, a role may be divided between more than one entity—for example where the medical provider 312 includes a local (e.g., relative to the user) general practitioner in cooperation with a non-local specialist. In another example, the medical provider 312, support personnel 314, and/or allergen test provider 318 may be a same person, persons associated with a same entity, and/or persons under the control of a same entity (e.g., employees, agents, suppliers, and/or contractors of an entity). In certain embodiments, the person in each role may vary during operations of the allergen test, for example over a period of days while the test is executed, and/or according to the state of the test (e.g., a first support person 314 may participate in patch placement, and a second support person 314 may participate in image capturing after patch removal). The role relationships described herein are representative examples for clarity of the present description, and are non-limiting to aspects of the present disclosure.

The example system depicts devices associated with the roles of the system—for example a user device 304 associated with a user 310, a medical provider device 306 associated with a medical provider 312, a support personnel device 308 associated with support personnel 314, and an allergen test execution controller 320 associated with an allergen test provider 318. The devices may each be a single device (e.g., a mobile phone, tablet, laptop, and/or desktop computer for a user device), distributed among several devices, and/or may be a first device at a first time and a second device at a second time (e.g., a user 310 utilizes a desktop computer to log in to an application provided by the allergen test execution controller 320 to answer questions and order an allergen patch kit 302, and uses a mobile phone to take and submit images for evaluation after patch removal). Additionally or alternatively, a given device may be embodied as multiple physical devices, for example at separate times and/or at a given time. For example, the user device may be a mobile phone having a mobile application stored thereon at a given time, and the user device may be a personal computer accessing a web application at another time. In another example, the user may interact with the system using both a mobile phone (e.g., taking and sending pictures and/or videos) and a personal computer (e.g., reading instructions, video conferencing with support personnel and/or the medical provider) at the same time, where the user device 310 includes both the mobile phone and the personal computer during such operations. The range of devices, including at different operating conditions and/or times, for any of the devices depicted are contemplated herein, and the description herein of specific devices and operating conditions is presented for clarity of the description, but is not limiting. Additionally or alternatively, a given device may serve as more than one role device, for example a given physical device may serve, at least during certain operating conditions for certain systems, as both the medical provider device 306 and the support personnel device 308.

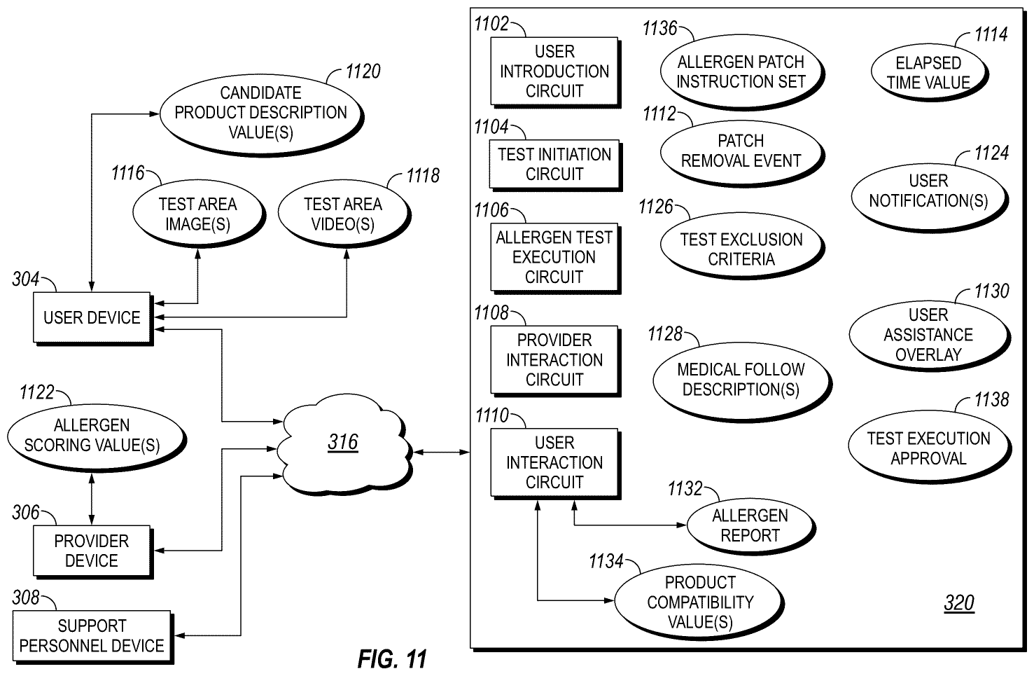

An example system includes the allergen test provider 318 providing an allergen patch kit 302 to a user. The allergen patch kit 302 includes a patch according to any embodiments of the present disclosure, having a selected group of allergens, which may be a general test group of allergens (e.g., a broad range of allergens for general diagnostic determination), a specific group of allergens (e.g., selected allergens according to some attribute of the user, such as age, gender, geographic location, occupation, exposure information, genetic information, one or more symptoms experienced, etc.), and/or may be a follow-up panel determined in response to a previously executed allergen panel test. In certain embodiments, the allergen patch kit 302 may further include one or more overlays, instructions, access instructions (e.g., for a mobile application, web based application, etc.), referral information (e.g., referring the user to a local medical provider), support information (e.g., contact information for support personnel), etc. An overlay may include a template, screen, or the like, configured to be placed over a test area (e.g., position on the skin of the user where the allergen patch is to be placed). In certain embodiments, an overlay may include a template, screen, or the like, configured to be placed over a screen of a mobile device (e.g., a mobile phone, tablet, or other device), for example allowing a light from the device to provide a configured illumination pattern over the test area, onto a visible space (e.g., wall, screen, white board, etc.) as a part of instructions and/or evaluation operations, or the like. In certain embodiments, the allergen patch kit may include a marker (e.g., a hypo-allergenic marker such as a gentian violet marker), which may be used to make marks on the patch, on the skin of the user in relation to the patch, on an overlay, and/or on the skin of the user in relation to the overlay. The example allergen patch kit 302 includes any aspects of an allergen patch test as set forth throughout the present disclosure, including at least: a patch with one or more allergen complexes and/or allergen panels; a positioning overlay usable before and/or to assist with ensuring that a skin test region is appropriate and/or proper placement of the patch; an evaluation overlay usable after patch removal to ensure proper imaging, assist in evaluation of the skin test region response, and/or to inform the user 310 about the results of the test; a marker to be utilized to mark alignment and/or orientation features, and/or to assist in ensuring that the patch is properly placed and/or replaced; and/or an instruction set (e.g., an allergen patch instruction set 1136, reference FIG. 11). In certain embodiments, the allergen patch kit 302 is at least partially positioned on a server and/or the allergen test execution controller 320, for example where the instructions, questions, evaluation interface, or other elements of the system are accessible through a web portal, mobile application, dedicated computer application, or the like. In certain embodiments, the allergen patch kit 302 includes instructions to access and/or execute elements of the system accessible through a web portal, mobile application, dedicated computer application, or the like, for example including web site information, app store information, login information, terms of use, licensing, or the like.

The example system further includes the support personnel 314 interacting with the user 310, for example using a support personnel device 308 in communication with a user device 304, for example to provide the user 310 with support in applying the allergen patch, for interacting with a mobile application or web application, for finding and/or interacting with a medical provider 312, and/or for interacting with the allergen test provider 318 (e.g., for replacing missing, lost, and/or defective aspects of an allergen patch kit 302, etc.). The example system includes a cross-device interaction component 316, which may be interactions between devices that occur in the context of a mobile application, web application, dedicated communications, video conferencing, etc. The cross-device interaction component 316 may include internet communications, intra-net communications, mobile communications, dedicated servers, or the like. The cross-device interaction component 316 may include some of these at a first time and/or between certain roles, and others of these at another time and/or between other roles (e.g., where the cross-device interaction component 316 includes mobile access to a mobile application at a first time, and web based interactions between the user device 302 and a server such as allergen text execution controller 320 over the internet at a second time). In certain embodiments, the cross-device interaction component 316 may change between interactions and/or within a given interaction, and the specific connectivity mechanisms between devices, roles, and/or operations is not limiting.

The example system further includes the medical provider 312 interacting with the user 310, and/or with the user device 302, for performing certain operations to plan, order, execute, and/or evaluate an allergen panel test. Example interactions of the medical provider 312 include referring the user to the allergen test provider 318 and/or ordering the allergen patch kit 302 from the allergen test provider 318. In certain embodiments, the medical provider 312 and the allergen test provider 318 may be the same entity, and/or aspects of the system described herein as performed by the allergen test provider 318 may be performed by the medical provider (e.g., the medical provider 312 ordering the allergen patch kit 302 to be shipped to the user 310). Further example interactions of the medical provider 312 include planning the allergen panel for the allergen panel test, including specifying one or more allergens to be included in the test, consulting with the allergen test provider 318 to select an appropriate test, providing the allergen test provider 318 with one or more patient attributes utilized to determine the allergen panel, or the like. Further example interactions of the medical provider 312 include evaluating one or more aspects of the test, for example confirming that pictures and/or videos are acceptable, evaluating results of the test (e.g., viewing the pictures, and determining allergic properties of the user), and/or consulting with the user 310 in regard to the results of the test (e.g., relaying likely cross-sensitivities, practical consequences of observed allergies, chemicals to be avoided, follow-up treatments and/or tests to be provided, etc.). In certain embodiments, certain activities of the medical provider 312 are performed in conjunction with and/or with the assistance of other aspects of the present system (e.g., where nominal allergen reports, evaluation parameters, picture evaluations, follow-up treatments, initial assessments, etc. are provided to the medical provider 312 by the allergen test execution controller 320), and/or performed iteratively with other aspects of the present system (e.g., where nominal outputs are provided by the allergen test execution controller 320 to the medical provider 312, updated at least in part by the medical provider 312, where the medical provider 312 then reviews the adjusted outputs resulting from updates, etc.).

The example system further includes the user 310 interacting with the system to participate, at least in part, in ordering, receiving, and/or executing aspects of an allergen panel test. The user 310 may engage the system for any reason, such as determining whether a suspected allergy is present, determining a baseline allergy response (e.g., performed for an industry specific purpose, such as an industry working with a sensitizing chemical), confirming that an allergy is not present in preparation for a specific event (e.g., before a surgery, dental procedure, medical treatment, exposure to a chemical, etc.), answering questions to determine the applicability of an allergen test and/or whether exclusionary criteria are present, referral to the system by a medical provider 312 (e.g., the same or a distinct medical provider from that depicted in FIG. 3). Example interactions of the user 310 with the system include ordering an allergen panel for an allergen panel test; confirming information to receive an allergen patch kit; receiving instructions and/or interacting with support personnel 314 to prepare for and execute a patch application; applying a patch; marking a patch; applying an overlay; marking an overlay; providing picture(s) and/or video(s) of a patch area; receiving a notification for a follow-up patch area view; providing follow-up picture(s) and/or video(s) of the patch area; and/or interacting with a medical provider 312 at various stages of the test execution process. Additionally or alternatively, interactions of the user 310 with the system include one or more of: receiving an allergen report; receiving communications such as follow-up tests and/or treatments; and/or providing a candidate product description (e.g., ingredient list, product title, link to ordering information, etc.) to the system.

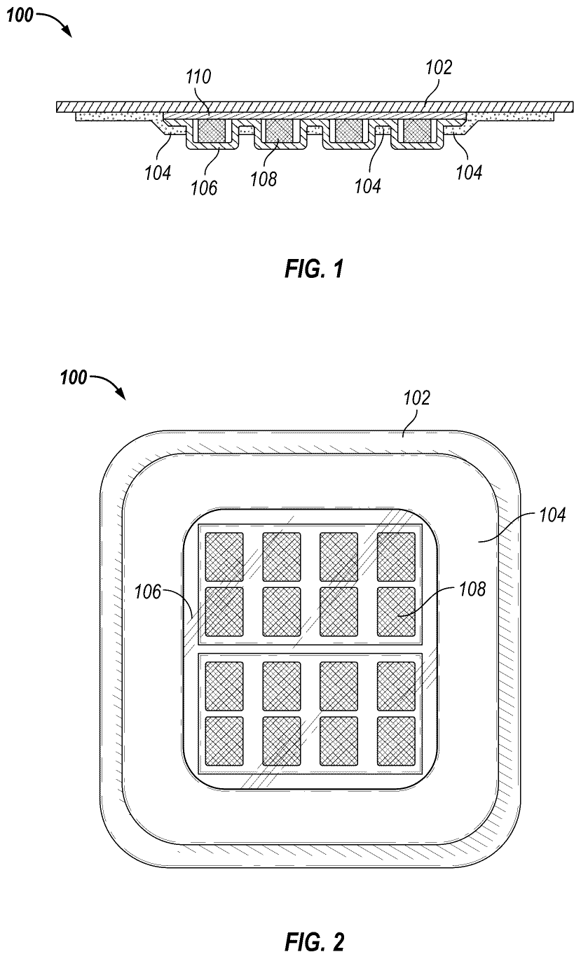

Referencing FIG. 1, an example allergen patch 100 having a number of aspects of the present disclosure is schematically depicted. The example allergen patch 100 is a multi-layer contact patch including a shield layer 102 releasably coupled to an adhesive layer 104. In the example of FIG. 1, the shield layer 102 is peeled off of the adhesive layer 104, and the patch 100 is applied to a target skin region, for example on a back, shoulder, thigh, upper arm, stomach, or other sufficiently sized and sufficiently flat area of the skin for contact exposure of allergens with the skin. In certain embodiments, the patch 100 is conformable to the area of the skin, and the target skin area need not be flat. In certain embodiments, the skin area to be utilized should not be a mobile area, such as an elbow or the like. The selected area may depend upon the type of adhesive utilized, the shape and/or conformability of the patch 100, and acceptable selected areas may be provided to the user in an allergen patch instruction set 1136 (e.g., reference FIG. 11 and the related description).

The adhesive layer 104 at least partially defines an allergen complex layer 106, for example partially or completely surrounding the allergen complex layer 106 (e.g., reference FIG. 2), and may further extend at least partially over the allergen complex layer 106 to secure the patch 100 in position during a test. In the example of FIG. 2, the adhesive layer 104 surrounds the allergen complex layer 106 completely, but the surrounding may be partial, for example with gaps around the perimeter of the allergen complex layer 106. In certain embodiments, the adhesive layer 104 may have portions that extend across the allergen complex layer 106, for example with a band or cross-member (not shown) extending across the allergen complex layer 106, for example between wells having an allergen complex 108 positioned therein.

The allergen complex layer 106 defines a number of wells having an allergen complex 108 positioned within at least some of the wells. For example, an allergen complex layer 106 may have a number of wells, with the allergen complexes 108 forming a selected allergen panel as set forth throughout the present disclosure. In the example, the allergen complex layer 106 may have a selected number of wells, or a large number of wells sufficient to support a selected allergen panel, where a given allergen panel may have the same number of allergens as the number of wells, a lower number of allergens than the number of wells (e.g., with some wells being empty and/or having a control material that does not include an allergen), and/or one less allergen than the number of wells, allowing for the utilization of one well to include a control material. The allergen complexes 108 each include an absorbent matrix and an allergen, where positioning of the patch on the target skin area places the allergen complexes 108 in contact with the skin, thereby causing an exposure of each allergen complex 108 for performing the allergen test. The absorbent matrix may be a gel or other material, and the allergens may be combined with the absorbent matrix at a selected concentration for a given well. In certain embodiments, the wells may be formed from a substrate material of the allergen complex layer 106, for example a metal foil (e.g., aluminum, stainless steel, or other non-irritating material) or plastic. In certain embodiments, the wells may be coupled to a substrate, for example with a flexible material (e.g., foil, fabric, film, etc.) between wells, and the wells coupled to the substrate. In certain embodiments, the wells are formed from an impermeable material, for example a material that does not significantly allow release of the allergen complex 108 within relevant time periods, including storage time, patch delivery time, and/or test execution time. In certain embodiments, the wells are lined with (e.g., inside the well) an impermeable material, and/or sealed with (e.g., outside the well) an impermeable material.

In the example of FIG. 1, the patch 100 includes a sealing layer 110 covering the wells of the allergen complex layer 106, and/or covering the entire allergen complex layer 106. The sealing layer 110 in the example is releasably coupled to the shield layer 102, and/or to the allergen complex layer 106. In certain embodiments, the sealing layer 110 may be positioned adjacent to at least a portion of the adhesive layer 104, and may be releasably coupled to the adhesive layer 104, or just positioned in proximity to the adhesive layer 104 without being coupled to the adhesive layer 104. The sealing layer 110, where present, prevents the migration of allergen complexes 108 between wells, the loss of allergen complex 108 material (e.g., evaporation of volatile compounds, etc.), prevents chemical changes (e.g., due to oxidation) of allergen complex 108 materials, and/or preserves the designed allergen complex 108 material (e.g., composition, volume, shape, etc.). Where present, the sealing layer 110 is removed after the removal of the shielding layer 102, and/or is removed with the shielding layer 102. In certain embodiments, the sealing layer 110 is not present, with either the sealing function being performed by the shielding layer 102, or the sealing function not being utilized in certain embodiments of the patch 100.

The example allergen complex layer 106 may include any number of wells, but generally may include at least two wells. The upper limit for the number of wells may be determined by the number of allergens to be included together within a given allergen panel, the size of the test skin area, the size of individual wells, the arrangement of wells of the allergen complex layer 106, or the like. In certain embodiments, the number of wells may be up to about 16 wells, up to about 32 wells, and/or up to about 48 wells. In certain embodiments, the number of wells may be provided as a multiple of 2, a multiple of 3, and/or a multiple of 4.

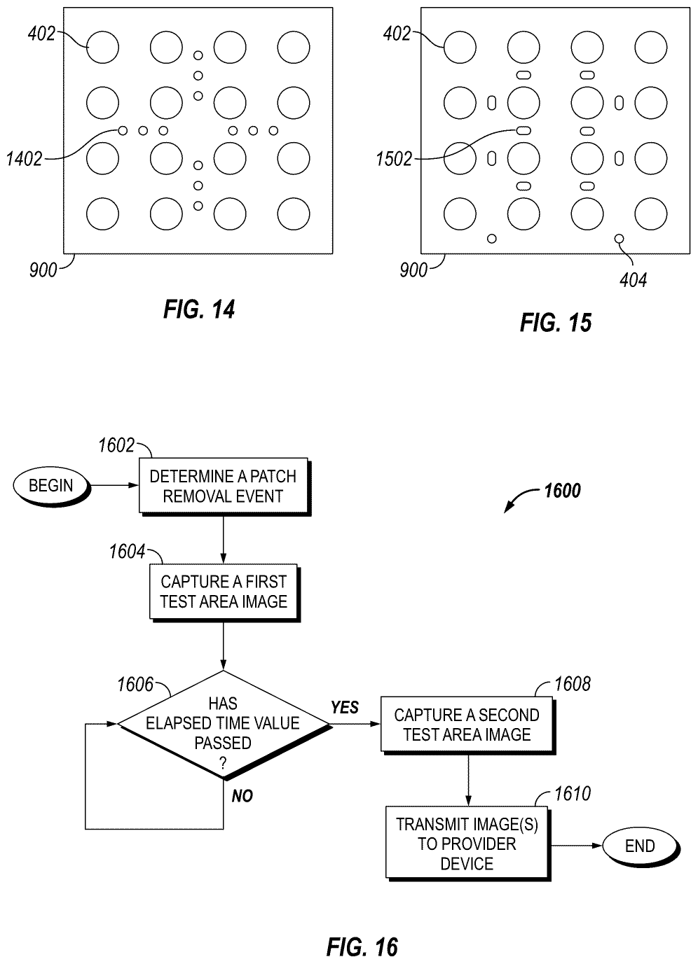

Referencing FIG. 2, an example patch 100 is depicted as viewed from the skin side (e.g., the side to be applied to the skin). In the example of FIG. 2, the shielding layer 102 is depicted as transparent, and the sealing layer 110 is omitted, to more clearly depict certain aspects of the patch 100. The example patch 100 includes the allergen complex layer 106 forming 16 wells having allergen complexes 108 positioned therein, and the adhesive layer 104 depicted having sufficient surface area in direct contact with the skin to secure the patch 100 for the duration of the test. In certain embodiments, the allergen patch 100 is placed on the skin for the test, and remains in place for a selected number of hours, for example 12 hours, 24 hours, 36 hours, 48 hours, 60 hours, or 72 hours. The allergen patch 100 is then removed, and either discarded or returned to a medical provider for analysis, quality control, or the like. In certain embodiments, the adhesive layer 104 may include features to ensure conformability of the patch 100 with the target skin area, for example with a perforation pattern (e.g., reference FIG. 14, which depicts an overlay with a perforation pattern 1402, but the adhesive layer 104 may include a similar perforation pattern to provide flexibility and/or conformity to the patch 100 as applied to the skin). The perforation pattern, where present, may include holes of any selected shape, positioned within the adhesive layer 104 to allow desired conformity, for example between wells of the allergen complex layer 106, and/or between groups of wells of the allergen complex layer 106.

In certain embodiments, the allergen complex layer 106 includes scores (e.g., areas that are thinner, marked, etched, etc.) in a scoring pattern to provide flexibility and conformity of the allergen complex layer 106 to the skin test region. For example, referencing FIG. 15, which shows a scoring pattern on an overlay but which is applicable to embodiments of the allergen complex layer 106, scores 1502 are provided in a pattern between adjacent wells, and/or between groups of wells, allowing the allergen complex layer 106 to conform to the skin test region and keep the allergen complexes 108 in contact with the skin during the test. In certain embodiments, the wells are scored, for example allowing flexibility of the well during the test to maintain contact of the allergen complex 108 with the skin.

An example patch includes where each one of the allergen complexes 108 includes a distinct allergen composition, which may be selected to form an allergen panel. An example allergen panel includes a selected group of allergens according to an expected population incidence rate, for example a group of allergens selected according to expected prevalence, sensitivity, historical occurrence, or the like for a population. The population for the expected population incidence rate may be determined according to any relevant criteria, including at least: occupation of members; geographic location of members; behavior of members (e.g., visits to certain locations, travel by defined methods, food preferences, sleep behaviors, etc.); place of birth of members; geographic trajectory of members including by age (e.g., geographic location at selected age ranges, travel between selected regions within a time period and/or at certain ages, etc.); demographic information of members; and/or according to an expressed profile by potential members of the population (e.g., responses to risk tolerance questions, determination of potential exposure events, correlated reactions to certain materials, products, or chemicals, etc.). An example allergen panel includes a selected group of allergens according to an exposure profile, for example relating to materials and/or chemicals determined to be potentially relevant to the exposure event (e.g., proximity to an event such as a chemical release or wild fire, participation in certain occupations, etc.). An example exposure profile is an industry specific profile, such as a mining panel, an agriculture panel, a manufacturing panel, a carpentry panel, or the like. In certain embodiments, an industry specific profile is related to a general industry, such as manufacturing, where common allergens related to the industry are included in the allergen panel. In certain embodiments, an industry specific profile is related to a specific industry, for example relating to particular chemicals or materials that are specific to a particular industry (e.g., a manufacturing environment where known and/or unusual chemicals may be utilized that might not be included in a general manufacturing panel). In certain embodiments, example and non-limiting allergen panels include one or more of: a metal panel (e.g., particular metals; metal compositions; alloys; common reaction products of particular metals; varying states of the metals such as particle sizes, surface area criteria, valence states, or the like; chemicals related to the processing of particular metals; etc.), a pediatric panel, a fragrance panel, an oil and gas panel, a refining panel, a carpentry panel, a health care panel, a hospital panel, a dye panel, a textile panel, surgical panel, a dental panel, a hairdresser panel, a specified exposure panel, and/or a food panel. The foregoing examples are non-limiting, and may be configured according to specific chemicals or materials likely to be present in related environments, breakdown products of these, estimated exposure rates and/or contact times, or the like. In certain embodiments, a selected allergen panel may include all or a portion of more than one allergen panel target, for example a combined metals and mining panel, depending upon the number of allergens to support each given panel, and the number of available wells to support allergen complexes 108 for a given patch. In certain embodiments, a control complex without an allergen is included on the patch, and/or included as a part of the selected allergen panel.

Referencing FIG. 4, an example overlay 400 and/or patch consistent with aspects of the present disclosure is depicted. The example patch 400 includes a substrate backing having an adhesive side (e.g., side 410 in the example of FIG. 4), having sufficient adhesive activity to maintain the patch in place after application (e.g., 2 days, or a few days). The adhesive side 410, in one example, is adhesive on the entire side, and/or adhesive in a pattern (e.g., surrounding the wells or allergen contact regions 402), with allergen contact regions 402 (or wells) positioned thereon. In certain embodiments, the adhesive side 410 forms the adhesive layer 104, and the allergen contact regions 402 form the allergen complex layer 106. The patch 400 may further include a shielding layer and/or sealing layer, for example as described in relation to FIGS. 1 and 2. As an overlay 400, the example of FIG. 4 may be made of plastic, paper, cardboard, or the like, and may be transparent or opaque.

In certain embodiments, the example of FIG. 4 may be an overlay, for example a template defining holes that parallel the allergen contact regions 402 of the patch, providing for a convenient patch placement and/or patch analysis guide. For example, a user may place the overlay, mark one or more places on the skin using the overlay 400 (e.g., marking an alignment and/or orientation feature 404), and utilize the marks to ensure that the patch is positioned properly before applying the adhesive layer 104 to the skin. The alignment and/or orientation features 404, where present, may be openings in the overlay 400 (e.g., with proper positioning ensured by making a mark on the skin test region visible through the overlay 400), marks on the overlay 400 (e.g., with marks on a transparent overlay being aligned with marks on the skin test region), or another feature such as a raised area of the overlay 400. In certain embodiments, the alignment and/or orientation features 404 ensure proper positioning of the overlay, proper analysis of the tested areas of the skin test region, and/or may allow imaging operations (e.g., by an allergen test execution circuit 1106, by a medical provider 312, by a technician and/or customer service representative 314, and/or by the user 310) to be properly performed, for example providing a baseline for de-skewing, scaling, and/or rotating an image, ensuring that the overlay 400 is in a proper position for imaging, and/or ensuring that an image of the skin test region has sufficient resolution, focus, and/or covers the planned test region.

In certain embodiments, the utilization of a placement overlay 400 allows for confirmation that the planned skin test region is sufficiently sized, that the patch will sufficiently conform to ensure contact of the allergen contact regions 402, and/or to allow a support person (e.g., medical provider 312 and/or customer service representative 314) to confirm proper placement of the overlay before the patch 400 is unsealed and positioned on the skin. In certain embodiments, alignment and/or orientation features 404 of the overlay may be mirrored in the patch, for example with matching holes or markings on the patch to allow the user to utilize the alignment and/or orientation features 404 for positioning of the patch. In another example, a user may place the overlay over the skin test region during analysis after the patch is removed. For example, the utilization of the overlay after removal of the patch allows for the user to ensure that the entire region is being properly imaged, to provide the user with information about which allergens were in each region (e.g., with labels on the overlay), and/or to provide a framework for a support person to discuss the test region and/or individual allergen regions with the user (e.g., to identify specific allergen or well locations with the user). In certain embodiments, the overlay 400 may be aligned with orientation and/or alignment features 404 to ensure proper positioning, for example where marks were applied by the user at the features 404 (e.g., during utilization of a placement overlay, during placement of the patch, and/or before removal of the patch), and/or where marks were applied by utilization of the patch itself (e.g., where the patch includes a marking material that marks the features 404 directly as a result of the placement and/or contact residence time of the patch).

An example patch substrate, for example of either the adhesive layer 104 and/or the allergen complex layer 106, is a polymer material having sufficient adhesive character on a first side (e.g., the skin facing side), with a treatment on the second side (e.g., the side away from the skin, or exposed side) such that the second side is non-adhesive, sufficiently water proof, etc. An example patch substrate is a material having appropriate structural characteristics (e.g., water proof, non-tearing, flexible within the range appropriate for the test area location and consistent with maintaining sufficient contact of the allergen complexes 108 to the skin, etc.), and with an adhesive layer 104 applied to the first side. In certain embodiments, the adhesive layer 104 may include a selected number of adhesive regions distributed over the first side of the patch. The allergen complexes 108 may be adhered to the first side of the allergen patch (e.g., at the wells of the allergen complex layer 106) via adherence of the allergen distributing matrix (e.g., a dried gel mix, or gel vehicle, which combined with the allergen may be referenced as an allergen complex), containment within the wells as applied to the skin, and/or utilizing another mechanism such as a dedicated adhesive for the allergen complex 108 within the well.

The allergen contact regions 402 of the patch may include an allergen complex 108 (e.g., an allergen distributed in a matrix, such as a dried gel matrix, at a selected concentration), a control region (e.g., an intentionally inert region, omitted allergen complex region, and/or control substance such as a matrix without an allergen component), and/or a logical region (e.g., a region that is available for placement of an allergen complex and/or reservation as a control region, but that may be merely blank for a given allergen patch, for example where fewer allergens are to be tested than the allergen patch is capable to accommodate). In certain embodiments, each allergen complex 108 may include a distinct composition, for example a separate allergen and/or control composition. In certain embodiments, one or more of the allergen complexes 108 may include a same material, for example to get more than one sample of a given allergen, to utilize more than one control region, etc. In certain embodiments, the allergen contact regions 402 of the patch correspond to the wells of the allergen complex layer 106. The allergen contact regions 402 of the overlay 400 may be openings to allow observation of the skin test region at the positions where the allergen complexes 108 were exposed to the skin during the test period. The matrix in which the allergen is distributed includes a material that is selected to be inert to allergens (e.g., does not react appreciably with the allergens for a selected time, such as a selected shelf life), that diffuses the allergen therein (e.g., providing a sufficiently uniform concentration profile throughout), that does not diffuse or creep outside the boundary (e.g., the allergen contact region defined for the particular allergen), and that is itself not a likely allergen. In certain embodiments, allergen complexes may utilize different distribution matrix materials, for example to account for distinct reaction characteristics, diffusion characteristics, molecular polarities, or the like.

An example allergen complex 108 is prepared in a procedure including mixing the allergen into the matrix material, which may be dried onto the allergen patch (e.g. within a well), and/or dried separately and bonded onto the allergen patch (e.g., within a well). The example procedure is illustrative and non-limiting. In certain embodiments, the matrix material may not be dried, and/or may be subjected to other processing appropriate to the material. Any allergen complex 108 adhered to the patch (e.g., the polymer) at the selected allergen contact region 402, and/or positioned within a well of the allergen complex layer 106 at the selected allergen contact region 402, is contemplated herein.

In certain embodiments, more than one control region may be present, for example to ensure that a reaction is not presented with regard to an adhesive or other component of the patch, and/or to a matrix utilized to bind and present allergens (e.g., a dried gel matrix). The example patch 400 includes a shape pattern having at least two distinct shapes (e.g., alternating circles and squares, in the example), where the shape pattern provides for case of orientation and/or alignment of the patch during operations to place the patch, to read the test area and/or reactions occurring in the test area, and/or to evaluate the treated area (e.g., an arm, back, stomach, or other region where the patch is placed) after removal of the patch. For example, the shape pattern can assist in positioning the patch (e.g., confirming that a selected shape is at the top), determining which allergen contact region had which allergen complex, and/or ensuring that images are oriented properly, have sufficient resolution and focus, and/or encompass the entire test area. The example patch includes a progression of two shapes as the shape pattern, but any number of shapes and/or shape progression is contemplated herein. In certain embodiments, uniquely shaped regions may be positioned at selected locations (e.g., three of the four corners of the patch), and/or regions having a distinct orientation (e.g., a rotated or partially rotated shape) to further enhance operations to orient, de-skew, align, and/or scale images of the test area. In certain embodiments, the shape of all allergen contact regions may be the same (e.g., a rectangle such as depicted in FIG. 2, or the shapes may be varied, sequenced, alternated, or configured in any other manner.

An example embodiment includes the example of FIG. 4 as a schematic representation of a patch 400, including orienting and/or aligning features 404, depicted as cross-shaped members at opposing corners of the patch in the example. In certain embodiments, alignment and/or orientation features 404 include an opening extending through the adhesive layer 104 and/or the allergen complex layer 106. In certain embodiments, alignment and/or orientation features 404 include marks made by the patch, for example an ink or other material that marks the alignment and/or orientation feature 404 due to placement of the patch. Any number, placement, shape, and/or orientation of orienting/aligning features 404 may be placed, for example with some features enhanced to improve orientation, alignment, scaling, and/or de-skewing operations. In certain embodiments, an orienting/aligning feature 404 may be visible on the second side of the patch (e.g., the side away from the skin), and/or with the patch in place (e.g., reference FIG. 7 and the related description), allowing for a user to utilize the features during placement operations of the patch, and/or allowing for confirmation of correct patch placement by a support personnel and/or a medical provider that may not be physically present with the user during placement. In certain embodiments, orienting/aligning features 404 may be holes extending through the patch (e.g., allowing for an external visual confirmation, and/or allowing for marking of the test area through the patch), an ink deposition region where the orienting/aligning feature leaves a mark on the test area after placement, and/or an externally visible mark, for example on the second side of the patch away from the allergen contact regions. In certain embodiments, the user may mark the orientation and/or alignment features 404, for example using a marker to mark the area through the patch (e.g., at the hole), overlay, or other aspect of the present disclosure having the orientation and/or alignment feature 404.

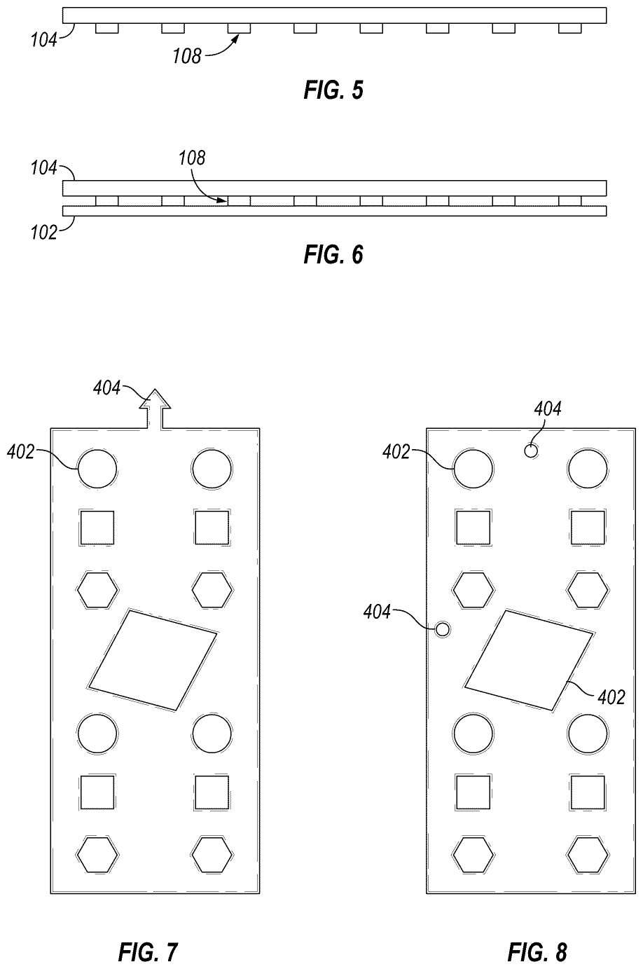

Referencing FIGS. 5 and 6, an example side view of a patch is depicted schematically. Referencing FIG. 5, the example patch includes the first side (e.g., adhesive side, and/or the side intended for direct contact to the test area) having allergen complexes 108 positioned thereon. The example allergen complexes 108 may be positioned within wells of an allergen complex layer 106. The example of FIG. 5 depicts the allergen complexes 108 as extending from the patch for clarity of the description, and are not shown to scale. Example allergen complexes 108 lay flat on the patch and do not significantly extend from the surface of the first side, and/or are positioned within wells of the patch. The actual geometry of an allergen complex 108 depends upon the amount and concentration of the allergen for a given allergen complex, the amount and deposition of the allergen complex on the patch (e.g., the amount of material and/or placement of the material wherein the allergen is distributed, such as a dried gel matrix), and the arrangement and configuration of wells (where present) of the patch.

Referencing FIG. 6, the example patch includes a protective layer 102, for example utilized during transport of the patch, and which is removed before placement of the patch. In certain embodiments, the protective layer 102 is a shield layer 102, for example as depicted in FIG. 1. The protective layer 102 covers active regions of the patch (e.g., allergen complexes, adhesive portions, control regions, etc.), and in certain embodiments may be co-extensive with the first side of the patch (e.g., covering the first side completely). The protective layer 102 may include features such as a removal tab for ease of separation of the protective layer 102 from the remainder of the patch. In certain embodiments, the protective layer 102 is a material that will not stick to the patch (e.g., to the adhesive layer 104 and/or to allergen complexes 108), that will not react with the allergen complexes (e.g., during storage and/or shipment, which may be extended periods up to a selected shelf life, for example 2 years), and that will protect the adhesive characteristic of the patch before removal of the protective layer and placement of the patch. Without limitation to any other aspect of the present disclosure, example and non-limiting protective layers may include release paper, a silicone coated paper, a Teflon™ coated paper, a wax paper, and/or Glassine. The examples are non-limiting. In certain embodiments, for example as depicted in FIG. 1, the patch includes a sealing layer 110 between the protective layer 102 and the allergen complexes 108, which may provide for better sealing of the allergen complexes 108, and/or case of installation, for example by allowing the user to work with the patch in a final form factor on the skin test region before unsealing the allergen complexes 108.

An example patch may additionally or alternatively be positioned in a further external package (e.g., a plastic wrap, foil pack, etc.). The additional external package may assist in preserving allergen concentrations and/or shelf life of the allergen patch, and/or protect the allergen patch during storage and/or transport.

Referencing FIG. 7, an example patch or overlay is depicted schematically, consistent with certain aspects of the present disclosure. The example of FIG. 7 includes a number of regions 402 which may be allergen complexes 108 (e.g., where FIG. 7 depicts a patch) and/or openings in the overlay at allergen contact regions (e.g., where FIG. 7 depicts an overlay), with variability in both the size and the shape of the allergen complex regions. Variability of allergen complex region sizes may be utilized, for example where indicated concentrations and amounts of an allergen provide for a distinct volume of allergen complex for those regions. The example patch includes an orientation feature 404, for example as an upward arrow providing a clear visual indication, visible from either side of the patch, indicating how to place the patch (e.g., including an instruction with the patch, and/or a marking on the patch, of “this side up”). In certain embodiments, the orientation feature 404 may assist the user to place the patch correctly, and/or assist the user to place an overlay correctly over the skin test region (e.g., after patch removal). The orientation feature 404 of the patch depicted in FIG. 7 may be left in place after placement of the patch, or may be removed (e.g., which may include perforations or another feature for ease of removal) after placement of the patch. In certain embodiments, an orientation feature 404 may be included as a part of the protective layer 102, and would thereby be removed as a part of the patch preparation and placement. In certain embodiments, the orientation feature 404 also provides a convenient aspect of the patch to case removal of the protective layer 102. In certain embodiments, the orientation feature 404 may be included as a part of another layer, such as the adhesive layer 104, and may be left in place with the patch, or removed after placement of the patch (e.g., tearing along perforations—not shown).

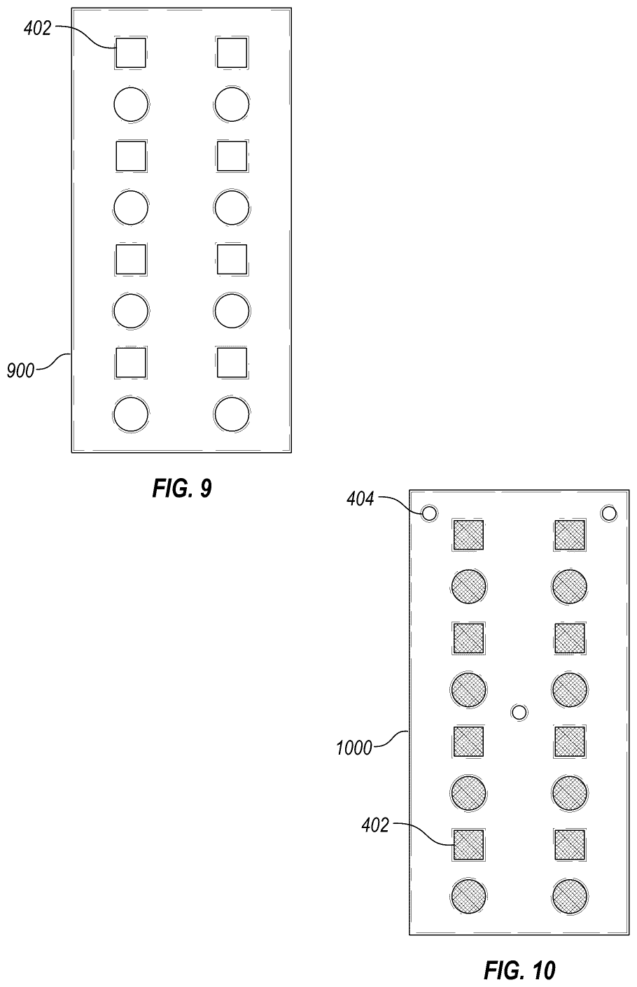

Referencing FIG. 8, an example patch or overlay is depicted schematically with orientation features 404 depicted that allow for confirmation of the patch placement both during patch placement operations and after removal, and/or with proper positioning of an overlay on the skin test region. The example orientation features 404 may be placed as holes through the patch (e.g., allowing for marking of the test area through the patch, and/or allowing for external confirmation of the patch placement), marks on one or both sides of the patch, as ink deposition regions that will mark the test area during the patch retention period of the test (e.g., the residence time of the patch on the skin before removal), and/or combinations of these. Example orientation features 404 may be placed as holes through the overlay, visible areas of the overlay (e.g., a transparent region, where a mark on the skin test region will be visible through the transparent region), and/or as marks on a transparent overlay (e.g., allowing for alignment of the marks with marks below on the skin test region). The example orientation features depicted in FIG. 8 may be combined with orientation features such as depicted in FIG. 7, or otherwise described throughout the present disclosure. In certain embodiments, selected allergen complex regions may be shaped and/or configured to act as orientation, alignment, scaling, and/or de-skewing features. The reference herein of certain features as orientation features or alignment features is not limiting, and a given feature may assist with orientation, alignment, proper positioning, or the like. Referencing FIG. 9, an example patch or overlay is depicted, consistent with aspects of the present disclosure. The example patch of FIG. 9 includes 16 available allergen complex regions, for example providing a patch capable to test 15 allergens with a control region.

Referencing FIG. 10, an example patch or overlay is depicted, consistent with aspects of the present disclosure. The example overlay may be a template (e.g., a paper, plastic, cardboard, stamped metal, or other material) with omitted regions over the allergen complex regions 402. The example overlay includes alignment/orientation features 404, for example making the orientation of the patch clearly visible to a remote user (e.g., a support personnel 314 and/or medical provider 312 viewing the test area through a camera, which they may not have control of), and/or to the user 310 (e.g., where the user may have a reverse view, or a poor view, of the test area, such as looking down at their upper arm, and/or viewing the test area in a mirror). The overlay may be utilized to mark the test area (e.g., after patch removal), to reference the test area allergen complex locations (e.g., in messaging with a support personnel and/or medical provider, and/or during discussion with the support personnel and/or medical provider; and/or when referencing a report, evaluation, or other communication that may reference patch locations). In certain embodiments, the overlay may have labels thereon, which may be general reference labels (e.g., A, B, C, . . . ), allergen codes and/or common names, chemical names, or the like. An example overlay includes a first set of labels on a first side, and a second set of labels on a second side (e.g., where the overlay is capable to be reversed and still be properly positioned, such as the example of FIG. 10 where the overlay has a vertical line of symmetry), thereby allowing the overlay to serve multiple audiences and naming conventions.

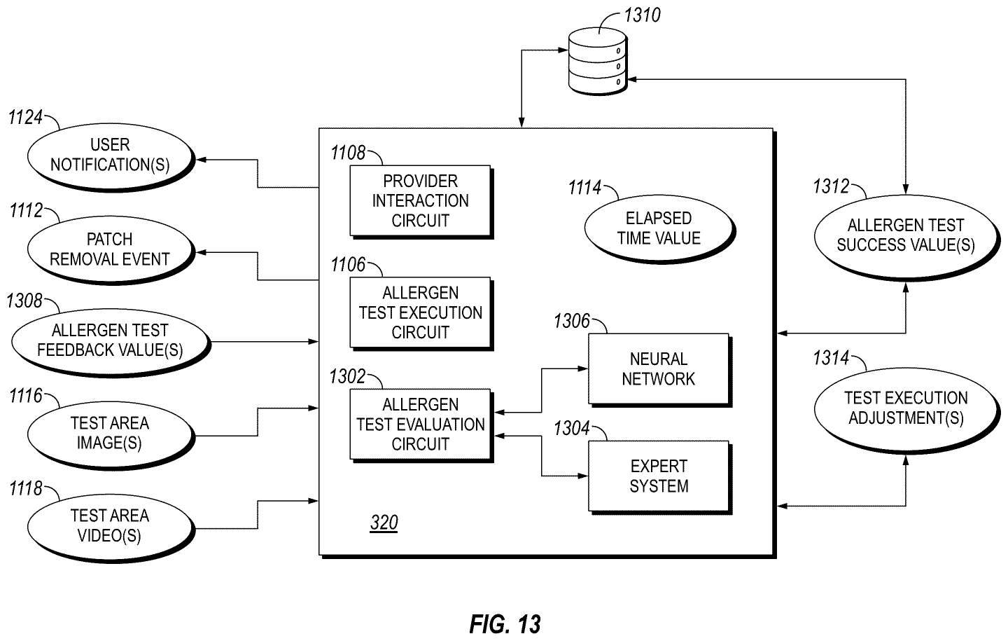

Further referencing FIG. 10, an example overlay may be a physical overlay, such as described in reference to FIG. 10 preceding, or a virtual overlay. For example, the overlay may be presented on a phone application (and/or any other computing device), for example as a real time overlay on a screen displaying a view of the test area (e.g., currently viewed on a camera) and/or as an overlay presented over a photo of the test area. The utilization of a virtual overlay allows for multiple operations of a device implementing the overlay (e.g., an allergen test execution controller 320, and reference FIG. 11 and the related description) such as, without limitation: providing a selected nomenclature for the test area (e.g., depending upon the role of the observer, such as the user 310 and/or a medical provider 312, responsive to a user selection, and/or responsive to an operating condition such as evaluation scores for each allergen complex region, a phase of the test, and the like); providing ancillary information such as evaluation scores, ingredients that are related to the region, and/or other allergens that are likely to be cross-sensitive to an allergen of a given region; selection of regions for further information; highlighting of a region for discussion (e.g., where a medical provider 312 highlights a region on the medical provider device 306, which is then highlighted on a user device 304 so the patient can follow along); accessing additional information related to the region (e.g., alternative pictures, videos, and/or earlier pictures from a given test); associating information related to the region (e.g., allowing the medical provider 312 and/or the user 310 to provide comments, which are then associated with the region); and/or limiting information access related to the region (e.g., allowing a medical provider 312 to make comments that are not visible to the user, that are selectively visible to another medical provider 312 such as a specialist, and/or allowing the user 310 to make comments that are not visible, etc.). Further operations of a device implementing the overlay, without limitation to any operations described herein, include: changing permissions for information (e.g., allowing a user, another medical provider, etc. to view previously hidden comments, and/or hiding previously visible comments); tagging specific regions or portions thereof (e.g., marking, circling, boxing out, etc. of specific portions of an image, and/or with associated comments); providing tentative comments and/or confirming comments as tentative; requesting an automated evaluation of a region (e.g., reference FIG. 13 and the related description); requesting an automated medical follow-up recommendation (e.g., reference FIG. 13 and the related description, and which may include adjustment, confirmation, submission, or other operations from the medical provider 312); accepting responses to automated messages and/or tagged messages (e.g., allowing the medical provider to incorporate, adjust, and/or remove automated recommendations; allowing the user to confirm and/or schedule a medical follow-up test, treatment, and/or doctor visit); and/or associating a portion of a video and/or conference session with a region (e.g., a time tag or other reference to a video depicting the test area; and/or a time tag or other reference to a recorded session, such as a commentary session by a medical provider and/or a conference session between the medical provider and another party such as the user or a specialist). The description includes providing an automated medical follow-up recommendation and/or an automated evaluation in response to a request from the medical provider, but other operations may include one or more of: providing automated medical follow-up recommendations and/or evaluations to the medical provider without a specific request; providing information about an automated evaluation to a user (with or without a request; and which may be marked as preliminary and/or include language indicating that the provided evaluation is not an actual diagnosis); and/or providing information about an automated medical follow-up recommendation to a user (with or without a request; and which may be marked as an example treatment that is not confirmed to be appropriate for the user).

An example overlay is a physical overlay that is provided for a device-such as a user device for a mobile phone screen and/or tablet. For example, an overlay template may be placed on the phone screen, where light from the device is then projected onto the test area. The light projected from the device may further project text or other information on the overlay, which will then be displayed at the appropriate location on the test area. The physical overlay for the device allows for adjustment of the display until the light projected through the overlay aligns with the test area. The utilization of a physical overlay for the device allows for many operations that are available on the purely virtual overlay, for example allowing the medical provider to highlight and/or comment on specific regions of the test area (e.g., illuminating them with a different color, changing text, etc.). In certain embodiments, permissions are enforced for viewing, commenting, and/or manipulating test information, for example by encrypting data communications between devices 304, 306, 308, and/or enforcing login access for personnel accessing or interacting with the controller 320 and/or medical or personal information of the user.

It can be seen that the operations of an overlay herein, including a physical overlay and/or a virtual overlay, and further including either a placement overlay (e.g., to assist in placing the patch) and/or an evaluation overlay (e.g., utilized on the skin test region after patch removal) can: improve the accuracy of the test by improving the confidence and success of initial patch placement; reduce the number of menus and interface interactions of the user and/or medical provider implementing the allergen test and evaluation; enhance the accuracy and improve the confidence of evaluation (e.g., enhancing the chance of an accurate evaluation, and improving confidence that an evaluation aspect is not missed); enhance information available to the user (e.g., facilitating communication with the medical provider and/or a support person, and providing information that may be relevant to their test); and provide for enhanced security and control of sensitive medical information.

Referencing FIG. 11, an example controller 320 configured to perform certain operations of the present disclosure is depicted. The example controller 320 is depicted as a single device for clarity of the description, but may be a distributed device, positioned on another device as a portion thereof, or combinations of these. For example the controller 320 may be embodied as the allergen test execution controller 320, but may additionally or alternatively be included, in whole or part, on one or more of the user device 304, the support personnel device 308, and/or the medical provider device 306. Additionally or alternatively, the controller 320 may be distributed across more than one of these, for example a system including more than one allergen test execution controller 320, and/or including one or more circuits or aspects of the controller 320 distributed across one or more devices. In certain embodiments, the configuration of the controller 320 may vary over time or operating conditions, for example with a portion of the controller 320 installed on a particular device 304, 306, 308 during certain operating conditions, and being removed after certain operations are completed (e.g., data and/or computer readable instructions may be stored on a user device and/or medical provider device, and may form a part of the controller 320, and/or be accessible to the controller 320, during a session between the user and the medical provider, and be removed upon the completion of the session).

The controller 320 includes a number of circuits configured to functionally execute operations of the controller 320. Each circuit is depicted as a single device for clarity of the present description, but a given circuit may be: a distributed device; provided as a part of another device; provided, in whole or part, as computer readable instructions stored on a memory (not shown); provided, in whole or part, as a logic circuit and/or hardware component configured to perform at least a portion of the operations of the respective circuit. A circuit, as utilized herein, may include any one or more of any actuator, sensor, or other component of any device herein (e.g., allergen test execution controller, user device, medical provider device, and/or support personnel device); any interface of a device and/or between devices; any hardware aspects involved in the execution of operations of the circuit and configured to perform and/or support such operations (e.g., an overlay, marker, and/or aspect of the patch); a mobile application which may be present on a cloud device and/or interfaces therewith; a web application and/or interface therewith; a cloud server; a cloud storage device; and/or a circuit may be in communication with any one or more of these.

The example controller 320 includes a user introduction circuit 1102 that provides an allergen patch execution instruction set 1136 to a user device, for example through a web-based interface and/or mobile application. In certain embodiments, the user introduction circuit 1102 provides the allergen patch execution instruction set 1136, in whole or part, as a physical instruction set, for example by determining information from the user by exercising an interface with the user device 304 to determine one or more aspects of the user, and providing at least a portion of the allergen patch instruction set 1136 as a part of an allergen patch kit 302. Example and non-limiting user information that may be relevant to the allergen patch instruction set 1136 includes, without limitation, any one or more of: user demographic information; user location information; user information relevant to exclusion criteria (e.g., user age, medications, recent medical events, contraindications, etc.); user information relevant to regulatory requirements (e.g., to ensure that required notifications, prohibitions, information protection, and/or other regulatory requirements are adhered to); user device information (e.g., device type, operating system, browser information, etc.); user access information (e.g., internet connectivity, mobile connectivity, phone information, e-mail addresses, login information, etc.); associated medical provider information (e.g., referring medical provider, primary medical provider, other medical providers that may provide information, access user information, and/or otherwise assist the user); and/or user physical information (e.g., to assist with patch sizing, selection, materials, etc.). In certain embodiments, the user information may include preferences of the user (e.g., access via a mobile application, web portal, phone calls, video conferencing, and/or in-person support such as with a local medical provider; shipping preferences; color preferences; overlay selection; etc.). In certain embodiments, the allergen patch instruction set 1136 may be provided, in whole or part, electronically such as through a mobile application, web portal, within an e-mail, accessible for viewing and/or download in an online format, via a third-party portal (e.g., a patient portal provided by a medical provider and/or a hospital), and/or provided to the user device from a dedicated application on the user device.

An example patch execution instruction set may include full instructions—for example and without limitation: telling the user how to apply the patch (e.g., including how to use a supporting placement overlay, how to clean the target area, how to orient the patch, how to mark the patch, etc.); how long to leave the patch in place; any care instructions relating to the patch and/or test area (e.g., including before, during, and/or after the test execution); any relevant conditions that should be noted to the application and/or to a medical provider, and/or conditions that may prohibit or affect the test (e.g., a sunburn, skin damage, medical conditions, certain medications, etc.); when to take images; the criteria for taking images (e.g., size, resolution, camera distance, file size, file format, etc.), and/or how to submit images (e.g., including on-line submissions, mobile application supported submissions, and/or physical address shipping locations); where to access assistance (e.g., a medical provider and/or support personnel); and/or referring the user to a local medical provider or other in-person resource. An example allergen patch instruction set 1136 includes a test execution schedule (not shown), for example indicating relative and/or absolute times for patch placement, patch removal, image capture operations, and/or follow-up operations such as evaluation, further testing, and/or medical treatments. The test execution schedule may include actual scheduled times and/or notifications (e.g., setting alert times, notification times, etc.) and/or estimated values for one or more of these (e.g., typical times for evaluation, follow-up, and/or treatment operations). The example allergen patch execution instruction set 1136 may include only a subset of these, and/or may include just access to support personnel and/or the medical provider (e.g., to ensure the user gets support before and/or during placement of the patch). The user introduction circuit 1102 may operate a mobile application, web based application and/or web portal, and/or device interface (e.g., operating a video conference between a support personnel device and the user device) to perform the related operations.

The example controller 320 includes an allergen test execution circuit 1106 that determines a patch removal event 1112 (e.g., responsive to a prompt from the user that the patch has been removed, responsive to a test area image where the patch is determined to be removed, etc.); captures a first test area image (e.g., as one of the test area images 1116) in response to the patch removal event 1112 (e.g., via instructions to and/or interactions with the user); provides a user notification 1124 in response to an elapsed time value 1114 from the patch removal event 1112 (e.g., providing a notification to the user that a prescribed time has elapsed, such as 2 days, a selected number of hours, etc.); and captures a second test area image 1116 in response to the elapsed time value 1114 (e.g., via instructions to and/or interactions with the user). The allergen test execution circuit 1106 may further provide a notification 1124 to the user when the patch is to be removed (e.g., after a predetermined time has elapsed since the patch was placed), records a time of patch placement, and/or captures images of the placed patch (e.g., to allow a support person and/or the medical provider to confirm proper placement). The allergen test execution circuit 1106 may further provide a notification 1124 of the time of the first test area image 1116 (e.g., notifying the user and/or medical provider, providing a time stamp with the first test area image 1116, and/or providing an elapsed time 1114 between the patch placement and the patch removal, between the patch placement and the first test area image capture, and/or between the patch removal and the first test area image capture). The allergen test execution circuit 1106 may further provide a notification 1124 of the time of the second test area image 1116 (e.g., notifying the user and/or medical provider, providing a time stamp with the second test area image, providing an elapsed time between the patch removal and the second test area image capture, and/or providing an elapsed time between the first test area image capture and the second test area image capture). An example allergen test execution circuit 1106 provides a communication interface between the user device 304 and the medical provider device 306 and/or support personnel device 308, for example providing for convenient support for the user, and confirmation of proper test execution, before the patch removal, first image capture, and/or second image capture. The operations of the allergen test execution circuit 1106 provide for, without limitation: case of the test execution by the user; confirmation of proper test execution; and/or the ability of the support personnel and/or medical provider to confirm and/or correct test execution (e.g., respond to an insufficient image) during a time period when the test can still be properly executed.

The example controller 320 includes a provider interaction circuit 1108 that transmits the first test area image 1116 and/or the second test area image 1116 to the provider device 306. Operations of the provider interaction circuit 1108 include transmitting the picture(s) during the capture operations by the user, and/or providing the picture(s) to a location accessible to the medical provider device 306 for access at a selected time. In certain embodiments, each of the first test area image 1116 and the second test area image 1116 are a high resolution image. A high resolution image, as used herein, should be understood broadly, and includes an image with sufficient lighting and pixel resolution for a medical provider 312 to determine the characteristics of allergen complex regions 108 of the test area (e.g., the area of the skin where the patch was placed, after removal of the patch). Without limitation to any other aspect of the present disclosure, characteristics of the allergen complex regions of the test area that are identified include: color of the region; the presence and characterization of erythema of the region; infiltration of the region (e.g., leakage into other areas); the presence and character of papules in the region; a vesicular reaction of the region; a bullous reaction of the region; an irritant reaction of the region; and/or a rash present in the region. It will be understood that an image that is a high resolution image may depend upon the environment (e.g., lighting, skin color), photo conditions (e.g., distance from the test area, motion of the photo device, etc.), the size of the allergen complex regions, and/or the goals of the test (e.g., detection of gross reaction, granularity of allergic determinations desired, simple baseline checking, etc.). One of skill in the art, having the benefit of the present disclosure and information ordinarily available when contemplating a particular allergen patch test execution system, can readily determine an image resolution that is a high resolution image. Certain considerations for determining whether an image is a high resolution image include, without limitation: the native resolution of the imaging device; the availability of multiple images (e.g., redundant views, additional views, and/or partial views) of the test area; the physical condition of the user/patient, including access to the test area, and/or operational capability with the imaging device; the ability of the medical provider and/or support personnel to directly interact with the user during image capture operations; the available lighting profile of users and/or ability of user devices to provide lighting; and/or the number, size, and/or arrangement of allergen complex regions 108.

An example allergen test execution circuit 1106 further performs at least one of a de-skew operation, an orientation operation, and/or a scaling operation on the first test area image 1116 and/or the second test area image 1116. Operations to perform a de-skew operation include correcting for changes in a depth of field of the image and/or squaring the image (e.g., where the image is taken from a significantly non-perpendicular view of the test area), and can be performed utilizing a known feature of the image (e.g., a size and shape of an orientation/alignment feature 404, and/or a clearly delineated allergen complex region 402) to correct the image shape and sizing distribution across the allergen complex regions 108. Operations to perform an orientation operation include rotating the image to provide a selected orientation (e.g., a portrait or landscape orientation), for example when the image is taken at a partially rotated angle (e.g., displaced a number of degrees from a desired orientation) and/or at an incorrect orientation (e.g., portrait rather than landscape, or vice versa). Operations to perform a scaling operation include re-sizing the image to provide a selected image size, for example to normalize for varying distance of the image from the test area, and/or for varying image size characteristics of the user device. In certain embodiments, operations performed to adjust the image (e.g., de-skew, scale, and/or orient) are combined with an operation to keep the original image as-taken, for example to allow the medical provider 312 to utilize both the raw and corrected images, and/or to allow for further processing of the image by an allergen test evaluation circuit 1302, neural network 1306, or expert system 1304 (e.g., reference FIG. 13 and the related description, for example to iteratively improve future image processing and/or test evaluation operations).

In certain embodiments, operations to perform de-skew, scaling, and/or orientation of the image(s) case the evaluation of the test area (e.g., providing a familiar orientation to the medical provider) and/or allow for selected application of a virtual overlay. In certain embodiments, one or more of the operations to de-skew, scale, and/or orient the image(s) are omitted, and the virtual overlay may be adjusted to be positioned on the image as captured, and/or as partially adjusted (e.g., where de-skew is performed, but not scaling, etc.). The operations to adjust the virtual overlay are performed similarly to the image adjustment operations, for example the virtual overlay is skewed to overlay properly on the image utilizing known features of the image, determining similar information that would be used to de-skew the image, for example allowing for correct marking of regions on the overlay.

An example allergen test execution circuit 1106 provides a user assistance overlay 1130, and captures the first test area 1116 after providing the user assistance overlay 1130. For example, the user assistance overlay 1130 may be placed on the screen of the user device 304, allowing the user to align the test area with the user assistance overlay, guiding the user to capture an acceptable image. The user assistance overlay 1130 may be aligned with the actual test area (e.g., the overlay displayed as skewed, scaled, and/or oriented according to the test area), and/or the user assistance overlay 1130 may be presented as an idealized overlay (e.g., with a squared, oriented, and scaled overlay), for example where the user moves the user device until the test area matches the idealized overlay (as much as possible), and/or where the idealized overlay reflects the sufficiency of the planned image 1116 (e.g., if the idealized overlay provides sufficient resolution, then the planned image 1116 is sufficient to be correctable for evaluation). The utilization of the actual test area for the overlay allows the user to move the device and observe the overlay (e.g., making it as square and/or large as possible) with feedback to the actual positioning of the user device. The utilization of an idealized overlay allows the user to optimize the image capture as much as possible. The utilization of both the actual test area for the overlay and the idealized overlay provides both benefits to the user, positioning the user device until the actual test area overlay matches the idealized overlay.

An example allergen test execution circuit 1106 photonically interrogates the test area, and determines that an acceptable image for the first test area (and/or the second test area) is available in response to the interrogating. Operations to determine the acceptable image is available include one or more of: determining one or more of de-skewing operations, orientation operations, and/or scaling operation to be performed on the image, and determining that these operations are within acceptable parameters (e.g., below a threshold correction value, etc.); determining the actual test area for the overlay and determining that it is within acceptable parameters (e.g., size, rotation, and squareness); determining the actual test area for the overlay and a difference value to the idealized overlay, and determining that the difference value is below a threshold; determining that an illumination level is acceptable; and/or determining that a focus level is acceptable. In certain embodiments, the allergen test execution circuit 1106 provides a user feedback value (e.g., as a test execution approval 1138, including a message, notification, sound, and/or haptic value such as a vibration) in response to determining the acceptable image is available. For example, when the user positions the user device in a manner that is expected to provide an acceptable image, the allergen test execution circuit 1106 may cause the feedback value (e.g., a quick double vibration) indicating the user can take the image. In certain embodiments, the allergen test execution circuit 1106 confirms that the captured image is an acceptable image, and causes the feedback value indicating the image taken was an acceptable image. The allergen test execution circuit 1106 may perform either one of the feedback operations (e.g., an acceptable image is ready to be taken, or an acceptable image was taken), or both. The utilization of a feedback value allows, without limitation, for the user to receive immediate confirmation that the test was properly executed, and/or allows the user to determine that that image was properly captured even where the image location (e.g., skin test location) and/or user physical limitations make it difficult for the user to ensure that a good image is being captured. Any one or more of the feedback values described may be provided as a user notification 1124 and/or a test execution approval 1138. In certain embodiments, the test execution approval 1138 is stored for access by the medical provider or a support person, and/or a notification is sent to the medical provider and/or support person when the test execution approval 1138 is determined.

An example allergen test execution circuit 1106 determines the test execution approval 1138 in response to a test geometry value (e.g., determining that a planned area of the skin test region is sufficiently sized, and/or sufficiently flat, to acceptably position the patch on the area), and/or in response to a test area clear value (e.g., determining that the test area is free from irritation, burns, wounds, and/or sufficiently clean, such that the test is expected to succeed, and/or the patch is expected to properly adhere to the test area). In certain embodiments, a provider interaction circuit 1108 provides a third test area image to the provider device 306, for example before the placement of the patch, allowing the provider to confirm the sufficiency of the planned test area, and/or allowing for operations of an allergen test evaluation circuit 1302, neural network 1306, or expert system 1304 to utilize the third test area image for iterative improvement operations on any aspect of the allergen test execution, including criteria for acceptable test geometry values, acceptable test area clear values, and/or to update or improve the allergen patch instruction set 1136.

An example allergen test execution circuit 1106 captures at least one additional test image 1116 (e.g., in addition to the first test area image and/or the second test area image), in response to the patch removal event 1112, an elapsed time value 1114, and/or an interaction with the user (and/or support personal and/or medical provider), and transmits the additional test image(s) to the provider device 306. Additional test images may be taken to provide for a 3D view of the test area (e.g., stereoscopic images), as a redundancy protection to ensure an acceptable image is taken, and/or at the request of the medical provider 312 interacting with the user 310. In certain embodiments, the additional image(s) 1116 may have the same criteria as the first image (e.g., squaring, scale, and/or orientation), and/or additional image(s) may have distinct criteria from the first image (e.g., different viewing angles, orientation, etc.). In certain embodiments, multiple images may be compiled to formulate a test image (e.g., where multiple lower resolution pictures are combined to form a single higher resolution image). In certain embodiments, the allergen test execution circuit 1106 provides distinct image acceptability checks and/or provides distinct virtual overlays to the user for one or more of the additional test images (e.g., images taken for a 3D view may be intentionally skewed slightly, and/or images may be taken at a different scale—for example to “zoom in” on an allergen complex region of interest).

An example allergen test execution circuit 1106 captures a video 1118 of the test area (e.g., at patch removal—or first test time; or after the elapsed time interval 1114—or second test time). The video 1118 may be captured in addition to the first test area image 1116 and/or second test area image 1116. An example allergen test execution circuit 1106 takes the first test area image 1116 and/or the second test area image 1116 as a selected image from the corresponding video 1118, for example utilizing an acceptable image from the video 1118 and alleviating the user from taking the still images. In certain embodiments, the allergen test execution circuit 1106 interrogates the video 1118 for acceptable images until an acceptable image is found, and provides a feedback value 1138 to the user. Accordingly, in certain embodiments, the user can take a video 1118 of the test area until the feedback value 1138 is received, indicating that an acceptable image 1116 is available (and/or a group of images that can be compiled into a larger image, and/or sufficient images to provide acceptable viewing of the allergen complex regions are available, etc.). In certain embodiments, a user assistance overlay 1130 may be presented on the user device during the video 1118 capture operations, for example providing an indication of when acceptable imaging and/or video data is available, regions of the skin test region (e.g., one or more allergen contact regions 402) where imaging data is acceptably captured and/or not sufficiently captured, etc. In certain embodiments, the utilization of a video 1118 and feedback value 1130 to provide images and acceptable image confirmation can assist users that have physical limitations (e.g., low mobility and/or low visual acuity), and/or users imaging test areas in a difficult location (e.g., on their back or shoulder blade area) to readily confirm that an acceptable image 1116 is provided for the first test area image and/or the second test area image. An example allergen test evaluation circuit 1302 (e.g., reference FIG. 13 and the related description) utilizes compiled images (e.g., from the first test area image, second test area image, available videos, and/or combinations of these) as a part of a training corpus of data, which may be utilized, without limitation, to identify areas for focus, further review, additional evaluation (e.g., taking a further image, which may include an image having distinct characteristics relative to the first and second test area images, a close-up of a portion of the first test area, etc.), and/or to adjust test execution parameters for subsequent allergen patch tests with the same or different users.

The example provider interaction circuit 1108 transmits the video(s) 1118 and/or selected images 1116 (e.g., standalone images, compiled images, and/or images captured from the video 1118) to the provider device 306. An example provider interaction circuit 1108 interprets an allergen scoring value 1122 from the provider device, for example using a scale indication such as depicted in Table 1. An example provider interaction circuit 1108 interfaces with the provider device 306 to get the allergen scoring value(s) 1122 for each region of the test area (e.g., for each allergen contact region 402).

In certain embodiments, an example provider interaction circuit 1108 populates an allergen scoring value 1122 for one or more of the allergen complex regions 402 of interest to the medical provider 312, which may be a default score value, preliminary score value, and/or an automated evaluation score. In certain embodiments, populate allergen scoring values 1122 may be determined from interactions with an allergen test evaluation circuit 1302, expert system 1304, neural network 1306, and/or using a rule-based determination (not shown) for example based on automatically identified features in the image (e.g., colors, features such as those described in Table 1, etc.). The provider 312 may replace or adjust populated scores, or confirm them. An example provider interaction circuit 1108 provides an indication to the provider 312 of scores that are populated and/or confirmed, for example color coding scores that are pre-populated and/or at default values, and adjusting the color coding in response to an adjustment by the provider 312 and/or a confirmation of the populated score by the provider 312. Example and non-limiting allergen scoring values 1122 include one or more of: a code or numerical indication, a description, and/or comments from the provider 312. An example provider interaction circuit 1108 associates an allergen scoring value 1122 with each allergen complex region 402, and/or with the control region (e.g., storing any comments from the provider associated with the control region).

Operations of the provider interaction circuit 1108 include, without limitation to any other aspect of the present disclosure, operating an interface to allow the provider 312 to interact with the provider device 306 and access imaging information, information about the user (e.g., any user information as described throughout, and/or as provided to the user introduction circuit 1102, user interaction circuit 1110, and/or allergen test execution circuit 1106), video information, test status information, notifications provided to the user, allergen report(s) 1132, or the like. In certain embodiments, the provider interaction circuit 1108 provides information to the provider device 306 by sending notifications and/or responsive data to the provider device 306, by making the information available to the provider device 306 to be accessed as desired (e.g., through a mobile application, web portal, dedicated application on the provider device 306, etc.), which may be stored on the provider device 306, on the controller 320, and/or on a server (not shown) at least selectively accessible to the controller 320 and/or the provider device 306.

An example allergen test execution circuit 1106 generates an allergen report 1132 in response to the allergen scoring value(s) 1122. An example allergen report 1132 includes one or more of, without limitation: allergen ingredient values (e.g., ingredients as listed in consumer products, food labeling, MSDS sheets, common names, etc., according to allergens having an indicated reaction from the test); allergen chemical description values (e.g., chemical names, common names, IUPAC names, etc., according to allergens having an indicated reaction from the test); and/or an offset allergen description value (e.g., related allergens that may be likely to have a reaction indication based upon the results of the test). In certain embodiments, cross-sensitive allergens may be listed as either an allergen or an offset allergen, for example based upon a likelihood that the allergen is a likely candidate. For example, if an allergen is virtually certain to have a cross-sensitivity based upon the test results, the allergen may be listed as an allergen or likely allergen, etc. In another example, if an allergen commonly includes a cross-sensitivity based upon the test results, or is known to have a potential cross-sensitivity, the allergen may be listed as a possible allergen, an allergen to check on through further testing, etc. The allergen report 1132 may be a populated report (e.g., based upon the allergen score values 1122 for each allergen complex region 402), and/or an adjusted report (e.g., with changes and/or confirmation provided by the medical provider). An example user interaction circuit 1110 provides the allergen report to the user device.

TABLE 1
Example allergen scoring values 1122

| Score | Description |
|---|---|
| − (0) | Negative reaction |
| ?+ | Doubtful reaction; erythema only |
| + (1+) | Weak (nonvesicular) positive allergic reaction; |
| | erythema, infiltration, and possibly papules |
| ++ (2+) | Strong (vesicular) positive allergic reaction; |
| | erythema, infiltration, papules, and vesicles |
| +++ (3+) | Extreme positive reaction; bullous reaction |
| IR | Irritant reaction |

The example scoring values 1122 are provided for illustration, and are not limiting. In certain embodiments, a scoring system may be defined for comparison and analysis of allergen patch test outcomes, for example utilizing numerical values, a scoring vector setting forth features of the test outcome (which may additionally be related to allergen contact regions, specific allergens, and/or patient attributes). The utilization of a scoring system may further allow for comparison across tests, across populations, across allergens, utilization in a deep learning operation, and/or standardization of test reporting.

An example user interaction circuit 1110 interprets a candidate product description value 1120. Example candidate product description values 1120 include an ingredient list associated with a product, a product identifier (e.g., SKU or other identifier), a link to a product description, an MSDS related to a product, etc. Operations to interpret the candidate product description value 1120 include the user providing the candidate product description value 1120 to the controller 320, whereby the allergen test execution circuit 1106 determines ingredient and/or chemical composition information, and determines a product compatibility value 1134 in response to the candidate product description value 1120 and the allergen report 1132. The version of the allergen report 1132 utilized to determine the product compatibility value 1134 may be the same or a different version from the allergen report 1132 provided to the user. For example, the allergen execution circuit 1106 may access a more complete, formal, or detailed allergen report (e.g., including full chemical names, a more complete list of cross-sensitivities, etc.) than the one provided to the user. The example candidate product description value 1120 may include an indication that the candidate product includes a known allergen, a likely cross-sensitive allergen, does not include a known allergen, and/or that the compatibility cannot be determined (e.g., unknown, ambiguous, and/or unavailable ingredients or composition, etc.). The user interaction circuit 1110 further provides the product compatibility value 1134 to the user device 304.

Operations of the user interaction circuit 1110 include, without limitation to any other aspect of the present disclosure, operating an interface to allow the user 310 to interact with the user device 304 and access and/or update imaging information, information about the user (e.g., any user information as described throughout, and/or as provided to the user introduction circuit 1102, user interaction circuit 1110, and/or allergen test execution circuit 1106), video information, test status information, notifications provided to the user, allergen report(s) 1132, or the like. In certain embodiments, the user interaction circuit 1110 provides information to the user device 304 by sending notifications and/or responsive data to the user device 304, by making the information available to the user device 304 to be accessed as desired (e.g., through a mobile application, web portal, dedicated application on the user device 304, etc.), which may be stored on the user device 304, on the controller 320, and/or on a server (not shown) at least selectively accessible to the controller 320 and/or the user device 304.

An example allergen test execution circuit 1106 determines a medical follow-up description 1128 in response to the allergen report 1132. For example, the allergen test execution circuit 1106 may access the allergen score values 1122 and allergens of the report 1132, and determine whether further testing, repeat testing, or other procedures are indicated. In certain embodiments, the allergen test execution circuit 1106 may access a treatment goals description (e.g., provided by the user and/or the medical provider—not shown), for example which may indicate that goal is to determine which allergens the user may have a sensitivity to, to reduce sensitivity impact on the user, and/or that the user may want to treat a sensitivity to reduce it if possible. Accordingly, an example medical follow-up description 1128 may further include one or more of: treatments indicated (e.g., medications, anti-inflammatories, and/or topical steroids); further allergen panels to perform; a follow-up test value; a repeat test value; an allergen panel description value; and/or a treatment plan description value (e.g., treatment descriptions and/or schedules, product changes to be made, lifestyle changes to be made, etc.). In certain embodiments, the allergen test execution circuit 1106 populates the medical follow-up description 1128, which may be adjusted or confirmed, in whole or part, by the medical provider 312. An example user interaction circuit 1110 provides the medical follow-up description 1128 to the user device 304, and/or provides a follow-up notification value 1124 to the user device 304 (e.g., to remind the user to schedule a test, order an allergen patch kit, etc.) in response to the medical follow-up description 1128. In certain embodiments, the user interaction circuit 1110 provides an interface for the user 312 to schedule an appointment with a medical provider, order an allergen patch kit, and/or provide an information update (e.g., to case communication of the user with a second medical provider for a second opinion, for consultation with a specialist, and/or to enhance the understanding of the user to their medical information) to the user in response to the medical follow-up description 1128.

An example controller 320 includes a test initiation circuit 1104 configured to operate a video conferencing interface, for example between the user and a support person, and/or between the user and a medical provider. In certain embodiments, the test initiation circuit 1104 operates the video conference to determine an allergen panel, allergen patch kit 302, answer preliminary questions such as a treatment goals description, and/or to determine whether test exclusion criteria 1132 are present that indicate that an allergen test is not indicated and/or that the test should be delayed (e.g., until the test exclusion criteria 1126 are cleared). In certain embodiments, the test initiation circuit 1104 operates the video conference to provide assistance for the user to initiate the allergen test, for example to perform patch placement operations. In certain embodiments, the test initiation circuit 1104 and/or the user introduction circuit 1102 provide test exclusion criteria 1132 to the user device, for example exercising a user interface on the user device to determine if the user is taking a medication that may prevent the test from providing reliable information (e.g., anti-inflammatory drugs, topical steroids, and/or immune-suppressing drugs), confirming that the user does not have a contraindicated medical condition, and/or confirming that the user has sufficient time and capability to reliably execute operations of the test (e.g., patch placement, wait time, removal operations, and imaging operations), connectivity (e.g., to utilize a mobile application, web portal, and/or data transfer from a dedicated application on the user device), and the like. In certain embodiments, the test exclusion criteria 1126 may be included, in whole or part, as a part of the allergen patch instruction set 1136. In certain embodiments, the test exclusion criteria 1126 may be checked by a support person or medical provider in a video conference or other messaging operated by the controller 320. In certain embodiments, the test exclusion criteria 1126 may be checked according to a user response, or lack of response, to questions provided by the allergen patch instruction set 1136, presented during an ordering operation for an allergen patch kit 302, and/or questions presented to the user device by the user interaction circuit 1110, user introduction circuit 1102, and/or test initiation circuit 1104 exercising a user interface of a mobile application, web portal, and/or dedicated application on the user device.

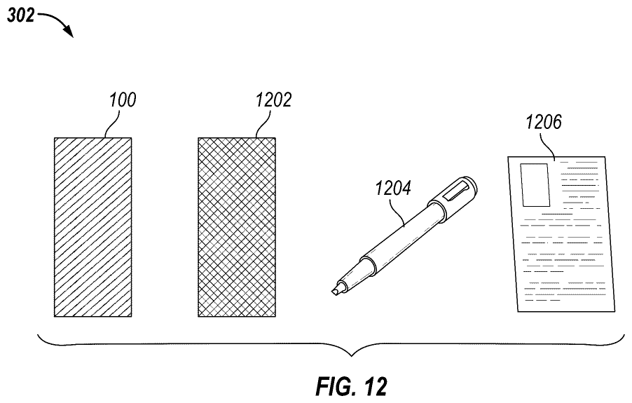

Referencing FIG. 12, an example allergen patch kit 302 is depicted schematically. The example components of the allergen patch kit are non-limiting, and a given allergen patch kit 302 may omit one or more described components, and/or include additional components, and/or more than one of the described components (e.g., providing a number of options for overlay(s), including full instructions 1136 along with a short version of the instructions and/or application access information, etc.). The example allergen patch kit 302 includes an allergen patch 100, which may be an allergen patch 100 according to any aspect of the present disclosure. In certain embodiments, the allergen patch 100 includes a shielding layer, an adhesive layer, and an allergen complex layer having a number of allergen complexes. In certain embodiments, the allergen patch 100 includes a sealing layer interposed between the shielding layer and the allergen complex layer, and positioned to seal wells of the allergen complex layer. The example allergen patch kit 302 includes one or more overlays 1202—for example an overlay 1202 for positioning over the test area (e.g., before and/or after removing the patch, and/or an overlay for positioning on a user device (e.g., over a screen of the user device, allowing the device to display information in the context of the overlay, and/or to project an image onto an object and/or onto the skin test region). In certain embodiments, an overlay 1202 may include any one or more of: a positioning overlay (e.g., allowing the user to check the skin test region, and/or mark the skin test region to assist in positioning the patch) and/or an evaluation overlay (e.g., assisting the user in properly imaging the skin test region, understanding the allergen report and/or medical provider comments, etc.). Any aspects of an overlay as described in the present disclosure are contemplated for embodiments of the allergen patch kit 302. An example allergen patch kit 302 optionally includes a hypo-allergenic marker, for example utilized to mark the test area (e.g., through the patch, on the patch, and/or near the patch; and/or in coordination with the overlay and/or a virtual overlay). An example hypo-allergenic marker includes a gentian violet marker, although any type of marker may be included in certain embodiments.

The example allergen patch kit includes an instruction set 1206 and/or an application access description. The instruction set 1206 may include any aspect of, or all of, an allergen patch instruction set 1136 as set forth throughout the present disclosure. Additionally or alternatively, the allergen patch instruction set 1136 may include reminders to the user, a listing of one or more test exclusion criteria 1126 (e.g., as a listing of excluding criteria, a questionnaire to be completed, and/or a questionnaire to assist the user in having appropriate information available when accessing a mobile application and/or web portal), descriptions that would be helpful to understand an allergen report 1132 and/or an allergy scoring value 1122, or the like. Without limitation to any other aspect of the present disclosure, an example instruction set 1202 includes one or more of: instructions for a user to apply the patch; a removal time; and one or more imaging times. The example access description, where present, includes login and/or registration instructions for one or more of a mobile application and/or a web application (e.g., allowing a user device to interact with one or more of the provider device, the support personnel device, and/or the allergen test execution controller), access user data related to the allergen patch test, access user medical data, access the allergen report, submit a candidate product description value, and/or perform any other operations or user interactions as set forth throughout the present disclosure.

Referencing FIG. 13, an example controller 320 configured to perform certain operations of the present disclosure is depicted. Without limitation to any other aspect of the present application, operations of the controller 320 example of FIG. 13 include operations to perform an allergen test, to provide an allergen report to the user and/or a medical provider, to perform a preliminary evaluation of the allergen test, and/or to iteratively improve performance of allergen test executions, evaluations, updates to the content and/or delivery of instruction sets 1136, updates to the content and/or delivery of notifications 1124, updates to the content and/or deliver of test exclusion criteria 1126, updates to the content and/or delivery of allergen patch kits 302, updates to the configuration and/or selection of allergen panels, updates to the configuration of overlays, updates to operating parameters for test execution (e.g., timing of allergen test steps, and/or interfacing operations with support personnel and/or medical providers), updates to imaging operations (e.g., picture instructions, resolution criteria, picture enhancement operations such as de-skewing, orienting, and/or scaling, updates to feedback operations for the user, etc.), and/or updates to the content and/or delivery of allergen reports 1132.

The example controller 320 is depicted as a single device for clarity of the description, but may be a distributed device, positioned on another device as a portion thereof, or combinations of these. For example the controller 320 may be embodied as a part of the allergen test execution controller 320, and/or as an allergen test improvement controller (not separately numbered), but may additionally or alternatively be included, in whole or part, on one or more of the user device 304, the support personnel device 308, and/or the medical provider device 306. Additionally or alternatively, the controller 320 may be distributed across more than one of these, for example a system including more than one allergen test execution controller 320 and/or allergen test improvement controller.

The controller 320 includes a number of circuits configured to functionally execute operations of the controller 320. Each circuit is depicted as a single device for clarity of the present description, but a given circuit may be: a distributed device; provided as a part of another device; provided, in whole or part, as computer readable instructions stored on a memory (not shown); provided, in whole or part, as a logic circuit and/or hardware component configured to perform at least a portion of the operations of the respective circuit.

The example controller 320 includes an allergen test execution circuit 1106, configured to perform operations including at least operations set forth for the allergen test execution circuit 1106 referenced in regard to FIG. 11 preceding. The example controller 320 further includes a provider interaction circuit 1108, configured to perform operations including at least operations set forth for the provider interaction circuit 1108 referenced in regard to FIG. 11 preceding.

The example controller 320 further includes an allergen test evaluation circuit 1302 configured to determine an allergen test success value 1312 in response to at least one of the patch removal event, the first test area image, and/or the second test area image. The example allergen test evaluation circuit determines the allergen test success value by performing at least one operation such as: determining that the patch removal event occurred within a specified time period; determining that a first test area image description (e.g., an image focus, resolution, illumination, coverage of the test area, time stamp of the image within a specified window, acceptable image enhancement operations, and/or an indication of success provided by the medical provider and/or support personnel received through an interaction with a corresponding device) indicating whether an acceptable first test area image was captured; determining that a second test area image description (e.g., an image focus, resolution, illumination, coverage of the test area, time stamp of the image within a specified window, acceptable image enhancement operations, and/or an indication of success provided by the medical provider and/or support personnel received through an interaction with a corresponding device) indicating whether an acceptable second test area image was captured; and/or receiving a test success description (e.g., as an allergen test feedback value 1308, e.g., from the medical provider 312 received through an interaction with the medical provider device 306) indicating whether the test was successfully executed (e.g., whether a clear allergen assessment can be made, whether acceptable images were captured within specified time periods, whether the patch placement and/or removal was successful, whether automated allergen scoring values 1122 were correct, etc.). The allergen test feedback value 1308 may be received actively, for example with the medical provider 306 confirming that aspects of the test were performed successfully, and/or passively, for example determined according to confirmation of automated evaluation values, according to an observation time of the medical provider 306 interacting with the images 1116, or the like. The example allergen test evaluation circuit 1306 may additionally or alternatively receive a test success description (e.g., as an allergen test feedback value 1308) from the user and/or from the support personnel, for example indicating whether corresponding portions of the test were understandable, properly executed, etc. In certain embodiments, the test success description includes one or more allergen test success values 1312, for example related to specific aspects of the test, and/or the allergen test execution circuit 1106 may determine allergen test success values 1312 from the test success description, and/or from operations performed during the test (e.g., based on one or more of time to execute imaging, adherence to time guidelines, completion time of information such as test exclusion criteria, whether marking was performed according to expectations, whether significant image enhancement and/or picture re-takes were performed, and/or whether an allergen patch kit 302 was complete, fully utilized, and/or required a re-order or other correction).

Example and non-limiting allergen test success values 1312 include one or more of: a first test area image description; a second test area image description; a patch placement description (e.g., positioned within a specified region; oriented within a specified range; aligned within a specified range; having acceptable placement parameters such as flatness and/or adhesion; and/or an elapsed time for placement operations of the patch); a patch orientation description (e.g., orientation and/or alignment within specified parameters); a patch marking description (e.g., a success value associated with the placement of related markings to the patch, and/or patch marking placed by the patch such as ink deposited from the patch to the skin); a predicted allergen response description (e.g., an indication of whether a predicted allergen response occurred according to the prediction, and/or a distance between the predicted allergen response and an exhibited response by the test); the predicted allergen response may include a first and second response, such as an initial response after patch removal, and a second response after an elapsed time such as when the second test area image is captured; where the predicted allergen response may be determined by a neural network 1306, expert system 1304, and/or the medical provider 306; and/or where the predicted allergen response may be determined in response to one or more of a patient attribute (e.g., age, biological sex, occupation, geographic location, allergen exposure profile, genetic marker, etc.) and/or an observed allergen response in an offset patient and/or population of patients (e.g., patients having a similar risk and/or exposure profile in some aspect); and/or a predicted patient response trajectory (e.g., a predicted progression of the patient response relative to one or more previous responses; the predicted patient response trajectory may include trajectories of the first and/or second response; where the predicted patient response trajectory may be determined by a neural network 1306, expert system 1304, and/or the medical provider 306; where the predicted patient response trajectory may be determined in response to one or more of a patient attribute (e.g., age, biological sex, occupation, geographic location, allergen exposure profile, genetic marker, etc.), an observed allergen response in an offset patient and/or population of patients, a treatment description (e.g., treatment information about the patient, such as treatments performed, and/or time values of the treatment(s)), and/or an exposure description (e.g., allergens the patient has been exposed to or likely exposed to, including acute and/or chronic exposure, environmental exposure, occupational exposure, surgical exposure, consumer product exposure, etc.).

An example allergen test evaluation circuit 1302 determines the allergen test success value 1312, and performs a test execution adjustment 1314 in response to the allergen test success value 1312. In certain embodiments, the allergen test success value 1312 corresponds to a first test event (e.g., the execution cycle of a first allergen test, including patch placement, marking, removal, first test area image capture, and/or second test area image capture), and the allergen test evaluation circuit 1302 performs the test execution adjustment 1314 on a subsequent test event (e.g., for the same or another patient/user). In certain embodiments, the allergen test evaluation circuit 1302 determines allergen test success values 1312 for a number of tests, for example tracking tests over time, and/or accessing a data store of allergen test outcome values 1310. Accordingly, the test evaluation circuit 1302 is capable to iteratively improve a trajectory of allergen test success values 1312. In certain embodiments, for example with the operation of a neural network 1306, the entire input space of the allergen test setup, delivery, execution, and evaluation, can be searched for correlation to the allergen test success values 1312, providing for improvements that may otherwise be missed even by a diligent medical provider 306 otherwise studying test outcomes. For example, presentation of instructions in the instruction set 1136, changes to a configuration of the allergen complex layer 106, such as well shape, depth, and/or materials, and/or changes to a presentation and/or wording of questions, may correlate to an increase in successful outcomes but are not likely to be checked, or checked efficiently, by previously known systems to perform allergen testing. In certain embodiments, any input available to the controller 320 may be investigated for correlations to successful outcomes, and adjustments 1314 may be provided for any output available to the controller 320, or available to personnel that can be reached by the controller 320 (e.g., the user, medical provider, support personnel, and/or allergen test provider), for example with the allergen test evaluation circuit 1302 providing an adjustment 1314 by providing a notification to the appropriate personnel advising of a change according to the adjustment 1314.

Example and non-limiting allergen success value(s) 1312 include one or more of a predicted allergen prevalence value (e.g., predicting an allergen prevalence for a population of users, for a geographic area, within an industry, etc.); a predicted trajectory of allergen prevalence values (e.g., predicting a progression and/or change over time of an allergen prevalence value); and/or a patient attribute correlation (e.g., clustering or grouping allergen response, allergen prevalence, trajectories of these, and/or predictions of these, based on one or more patient attributes).

Example and non-limiting test execution adjustments 1314 include one or more of: adjusting an allergen panel (e.g., adjusting allergens provided on a patch, and/or a number of allergens to be provided on the patch); adjusting an allergen concentration value (e.g., adjusting a volume of one or more allergen complexes, and/or an amount of a given allergen in one or more allergen complexes); adjusting an alignment feature and/or an orientation feature of an allergen patch (e.g., the size, shape, position, type-such as a hole through the patch, a tab extending from the patch, a user-marked location, a patch-marked location, etc.); adjusting an alignment feature and/or an orientation feature of an overlay; adjusting an elapsed time value (e.g., a time between patch placement and patch removal, and/or a time between a first test area image capture and a second test area image capture, and/or including a number and/or timing of image capture events); adjusting a de-skew, alignment, and/or scaling operation (e.g., increasing or decreasing a correction applied; a target range of acceptable skew/alignment/scaling of an image; adjusting a type of virtual user overlay, such as an idealized overlay and/or an actual test area overlay; adjusting a virtual user overlay parameter, such as a color, line-type, constraint, etc. for a virtual user overlay; adjusting a physical overlay (e.g., sizing, labeling, alignment features, material, etc.); adjusting a user device display response to an overlay (e.g., adjusting a light type, light amount, text location and/or capability, etc.); adjusting an image lighting parameter (e.g., user device position target; a lighting operation and/or acceptable lighting parameter; a focus requirement; a skew, scaling, and/or orientation requirement, e.g., instructing the user to adjust image size, distance, and/or user device position during capture of an image); an acceptable image determination value (e.g., adjusting parameters utilized to determine whether an acceptable image is available for capture); adjusting an image type parameter (e.g., adding, changing, and/or removing an image type such as multiple images for a given capture, video taken for a given capture, 2D versus 3D imaging, etc.); adjusting an image processing parameter (e.g., operations to crop and/or compress one or more images, including potentially adjusting a compression algorithm, such as changing between a lossless or lossy compression algorithm, and/or adjusting a level of compression of an image or video compression algorithm); adjusting a user feedback value (e.g., a feedback type such as sound, light, messaging, and/or vibration; a feedback parameter such as a duration, number of instances, amplitude, or other parameter related to a feedback such as a feedback indicating that the user device is properly positioned for an image, and/or that an acceptable image has been captured); adjusting a gel vehicle utilized for one or more allergen complexes (e.g., a composition of the gel; a method of formulating the gel such as mixing operations, drying time, etc.; and/or a volume and/or placement of the gel on the patch and/or within a well); adjusting a user device position value for capturing the first test area image and/or second test area image (e.g., a distance, orientation, acceptable skew value, etc.); adjusting an allergen patch application instruction (e.g., the presence of an allergen patch application instruction; a content of the allergen patch application instruction; and/or a font and/or placement of text aspects of the allergen patch application instruction); adjusting an allergen application access instruction (e.g., the presence of an allergen patch application instruction; a content of the allergen patch application instruction; and/or a font and/or placement of text aspects of the allergen patch application instruction); and/or adjusting an arrangement, spacing, size, and/or shape of allergen contact regions of a subsequent allergen patch, which may include allergen-specific adjustments (e.g., changing parameters for a specific allergen, and/or enforcing spacing between two or more selected allergens).

An example provider interaction circuit 1108 interprets an allergen test feedback value 1308 from the provider device 306—for example operating an interface with the provider device 306 allowing the medical provider 312 to provide the allergen test feedback value 1308. The provided allergen test feedback value 1308 may be utilized for any one or more of: utilization as the allergen test success value 1312 to determine a test execution adjustment; utilization of test parameters and the allergen test feedback value 1312 as an input for the allergen test outcome values 1310 of a data store; and/or to inform the determination of at least a portion of the allergen test success value 1312.

An example allergen test evaluation circuit 1302 is configured to train on a corpus of data including a number of allergen test outcome values and/or allergen test success values 1312. An example corpus of data includes allergen test outcome values provided on a data store accessible to the allergen test evaluation circuit 1302. The corpus of data may include actual data (e.g., from a number performed allergen patch tests), configured data (e.g., example data having a selected range and/or occurrence rate of input values, such as patient attributes, allergen panels, exposure profiles, images, elapsed time values, etc.; and/or a selected range and/or occurrence rate of output values, such as allergen responses, allergen scoring values 1122, and/or allergen test success values 1312.

An example allergen test evaluation circuit 1302 operates a neural network 1306 to determine the allergen test success value 1312, the test execution adjustment 1314, and/or an allergen scoring value 1122. For example, a neural network 1302 may utilize the corpus of data for training the determinations, and/or may adapt to ongoing allergen test events. An example neural network 1302 has authority to adjust correlations between input values and output values, including adjusting the strength of the correlations, the removal of inputs having lower predictive value, and/or the addition of inputs having higher predictive value.

An example allergen test evaluation circuit 1302 operates an expert system 1304 to determine the allergen test success value 1312, the test execution adjustment 1314, and/or an allergen scoring value 1122. For example the expert system 1304 may include a model for determining the allergen test success value 1312, the test execution adjustment 1314, and/or the allergen scoring value 1122. The model may include correlating aspects of test input values (e.g., including any inputs set forth herein, including patient attributes, imaging attributes, elapsed time values, etc.) to output values. In certain embodiments, the model may be tuned by an expert and/or a group of experts (e.g., a medical provider and/or group of medical providers, and additionally or alternatively support personnel—for example to advise on successful test execution operations). In certain embodiments, the allergen test evaluation circuit 1302 further operates a neural network 1306 in concert with the expert system 1304—for example to determine one or more input values (e.g., image quality or parameters, and/or adjustment of inputs utilized in the model) for use by the expert system 1304. An example system includes a neural network 1306 and/or expert system 1304 operating to populate allergen scoring value(s) 1122, descriptions and/or instructions, and/or medical follow-up descriptions 1128, for example as set forth in relation to FIG. 11 preceding. An example system includes a neural network 1306 and/or expert system 1304 predicting an allergen response, an allergen prevalence, and/or trajectories of these. An example system includes a neural network 1306 and/or expert system 1304 determining and/or predicting a cross-sensitivity value (e.g., a second allergen that is likely to show a response in light of a first allergen showing a response).

Example allergen panel(s) may be selected to support baseline tracking—for example to track allergen response and/or sensitivity development for a user associated with an industry, activity, and/or application that may cause exposure to a sensitizing chemical (e.g., epoxy, resin, formaldehyde, dye, fragrance, preservative, topical medication, antibiotic, sulfates, and/or metal such as Cr, Au, Ni, Cd, etc.). An example allergen panel is selected in response to an allergen prevalence in a population, including an observed and/or predicted prevalence, and which may include a determination of the prevalence based on one or more of: a selected context (e.g., user occupation, location, hobbies, activities, etc.); a consumer product prevalence (e.g., beauty products, skin care products, hair products, grooming products, and/or food products); and/or an event based determination (e.g., natural disasters; industrial incidents; weather events; and/or wild fires including a terrain composition of an region related to a fire).

An example allergen panel is depicted in Table 2. The example allergen panel is representative of a panel of prevalent allergens related to a typical user exposed to normal consumer products and environment. The allergen panel of Table 2 is not limiting in any aspect, including the number of allergens provided, the specific composition of selected allergen types, and/or the concentration ranges presented for allergens. The prevalence values depicted in Table 2 are representative examples for a nominal population, and are not limiting to the present disclosure. The prevalence values depicted in Table 2 are an illustrative prevalence of allergen sensitivity in a given population, and do not correspond to a prevalence of an allergen exposure in the population. Without limitation to any other aspect of the present disclosure, both prevalence values are contemplated herein.

TABLE 2
Example allergen panel

| Allergen | Concentration | Prevalence |
|---|---|---|
| Nickel Sulfate | 5% pet [2 mg/cm2] | 17.5% at 2.5% test, |
| | | closer to 30% at 5% |
| | | pet |
| Methylisothiazolinone [MI] and | 0.2% aq of MI and 0.015% aq of MCI | Estimated at 17% |
| Methylchloroisothiazolinone | [total 0.215% aq] | |
| [MCI] mix | | |
| Fragrance Mix I | 8% pet, 1% of each of 8 allergens: | 11.3% |
| | Geraniol, Cinnamaldehyde, | |
| | Hydroxycitronellal, Cinnamyl alcohol, | |
| | a-Amylcinnamaldehyde, Isoeugenol, | |
| | Eugenol, and Oak moss [3.2 mg/cm2] | |
| Fragrance Mix II | 14% pet: coumarin 2.5%, | 5.3% |
| | hydroxymethylpentylcyclohexane | |
| | carbaldehyde (lyral) 2.5%, citronellol | |
| | 0.5%, farnesol 2.5%, citral 1%, alpha- | |
| | hexylcinnamic aldehyde 5%, contains | |
| | 10% paraffin, hard | |
| Formaldehyde | 2% aq [0.6 mg/cm2] | 8.4% |
| Balsam of Peru | 25% pet [10 mg/cm2] | 7.0% |
| [myroxylon pereirae resin] | | |
| 4-Phenylenediamine base | 1% pet [0.4 mg/cm2] | 6.4% |
| Propylene Glycol 100% | 100% | 4.0% |
| Lanolin Alcohol | 50% pet [?20 mg/cm2] | 4.1% |
| Quatemium-15 | 2% pet [0.8 mg/cm2] | 3.6% |
| 2-Hydroxyethyl methylacrylate | 2% pet | 3.4% |
| [HEMA] | | |
| Methyldibromo | 2% pet | 3.5% |
| Glutaronitrile/Phenoxyethanol | | |
| Cocamidopropyl betaine | 1% aq | 1.6% |
| Paraben mix | 16% pet: [mix of butyl-, ethyl-, | 0.6% |
| | methyl-, and propyl-parabens 4% each | |
| | [6.4 mg/cm2] | |
| Iodopropynyl Butylcarbamate | 0.5% pet | 3.9% |
| Control region | Omitted | N/A |

Referencing FIG. 16, an example procedure 1600 for providing test information of an allergen test to a medical provider is schematically depicted. The example procedure 1600 may be performed, in whole or part, by any controller, circuit, or aspect of the present disclosure, including at least operations of a controller 320 such as depicted in FIGS. 11 and/or 13. The example procedure 1600 includes an operation 1602 to determine a patch removal event, an operation 1604 to capture a first test area image, and an operation 1606 to determine whether an elapsed time value has passed since the patch removal event and/or the first test area image capture. In response to operation 1606 indicating NO, the example procedure 1600 continues to monitor the elapsed time. In response to operation 1606 indicating YES, the example procedure 1600 includes an operation 1608 to capture a second test area image, and an operation 1610 to provide the images to a medical provider device.

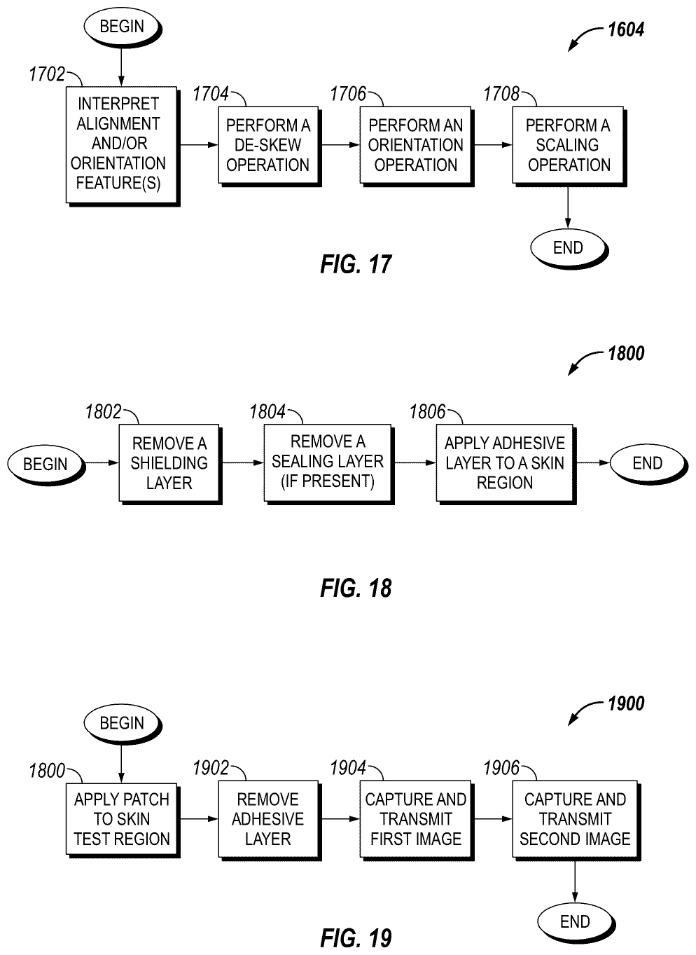

Referencing FIG. 17, an example procedure 1604 to capture and/or enhance a test area image is schematically depicted. The example procedure 1604 may be performed for the first test area image, the second test area image, and/or for any additional test area images, for example as set forth throughout the present disclosure. The example procedure 1604 includes an operation 1702 to interpret alignment and/or orientation features (where present), which may additionally or alternatively include interpreting a size and/or shape of allergen contact regions of an overlay and/or on the skin test area. The example procedure 1604 includes an operation 1704 to perform a de-skew operation, an operation 1706 to perform an orientation operation, and/or an operation 1708 to perform a scaling operation of the image. In certain embodiments, the image after processing 1604 is provided to a medical provider device.

Referencing FIG. 18, an example procedure 1800 for applying an allergen patch is schematically depicted. The example procedure 1800 includes an operation 1802 to remove a shielding layer, an operation 1804 to remove a scaling layer (if present), and an operation 1806 to apply an adhesive layer of the patch to a skin test region.

Referencing FIG. 19, an example procedure 1900 to perform an allergen test is schematically depicted. The example procedure 1900 includes an operation 1800 to apply an allergen patch to a skin test region. The example procedure 1900 further includes an operation 1902 to remove the adhesive layer from the skin test region (e.g., removing the patch from contact with the skin), an operation 1904 to capture and transmit a first image of the skin test region (e.g., immediately after, or soon after, removal of the patch), and an operation 1906 to capture and transmit a second image of the skin test region. In certain embodiments, operation 1906 is performed within a predetermined time range after operation 1904, for example between 24 to 48 hours, or between 12 to 72 hours, after operation 1904. In certain embodiments, operation 1904 is performed within a predetermined time range after operation 1800, for example between 24 to 96 hours, between 12 to 72 hours, or between 48 to 96 hours after operation 1800.

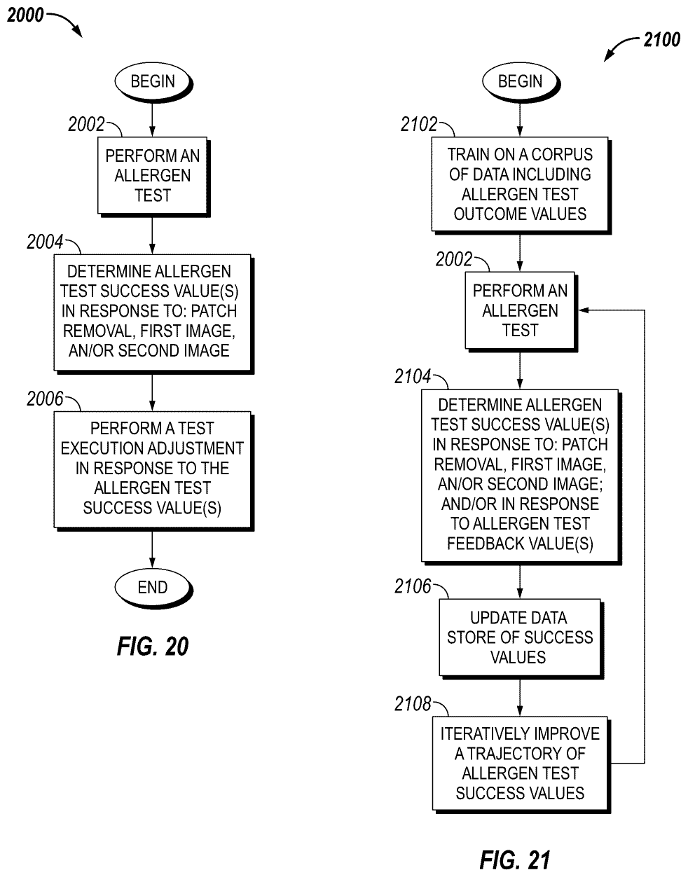

Referencing FIG. 20, an example procedure 2000 for evaluating and improving an allergen test execution operation is schematically depicted. The example procedure 2000 may be performed, in whole or part, by any controller, circuit, or aspect of the present disclosure, including at least operations of a controller 320 such as depicted in FIGS. 11 and/or 13. The example procedure 2000 includes an operation 2002 to perform an allergen test, an operation 2004 to determine allergen test success value(s) in response to the test, including in response to parameters such as a patch removal event, a first image, and/or a second image. The example procedure 2000 includes an operation 2006 to perform a test execution adjustment in response to the allergen test success value(s).

Referencing FIG. 21, an example procedure 2100 for iteratively improving a trajectory of allergen test success values is schematically depicted. The example procedure 2100 may be performed, in whole or part, by any controller, circuit, or aspect of the present disclosure, including at least operations of a controller 320 such as depicted in FIGS. 11 and/or 13. The example procedure 2100 includes an operation 2102 to train a learning component (e.g., a neural network, expert system, rules-based system, etc.) on a corpus of data including allergen test outcome value(s), which may further include test input values such as instruction data set information, test execution information, user information (e.g., which may be anonymized), etc. The example procedure 2100 includes an operation 2002 to perform an allergen test, and an operation 2104 to determine allergen test success value(s) in response to the test, including potentially in response to allergen test feedback value(s) as set forth throughout the present disclosure. The example procedure 2100 includes an operation 2106 to update a data store of allergen test success values (e.g., data store 1310), and an operation 2108 to iteratively improve a trajectory of allergen test success values (e.g., by selectively performing test execution adjustment(s)).

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer readable instructions described above. All such permutations and combinations are contemplated in embodiments of the present disclosure.

What is claimed is:

1. A multi-layer contact patch, comprising:
a shield layer releasably coupled to an adhesive layer;
the adhesive layer at least partially defining and fixably coupled to an allergen complex layer;
the allergen complex layer defining a plurality of allergen complex cavities; and
wherein at least a subset of the plurality of allergen complex cavities comprise an allergen complex positioned therein, each allergen complex comprising an absorbent matrix and an allergen.

2. The multi-layer contact patch of claim 1, further comprising a sealing layer interposed between the shield layer and the plurality of allergen complex cavities, wherein the sealing layer is releasably coupled to the shield layer and the plurality of allergen complex cavities.

3. The multi-layer contact patch of claim 1, wherein the allergen complex layer defines between 2 and 48 allergen complex cavities, inclusive.

4. The multi-layer contact patch of claim 1, wherein the allergen complex layer defines 16 allergen complex cavities.

5. The multi-layer contact patch of claim 1, wherein the adhesive layer further comprises a perforation pattern.

6. The multi-layer contact patch of claim 5, wherein the perforation pattern comprises a plurality of perforations between adjacent ones of the plurality of allergen complex cavities.

7. The multi-layer contact patch of claim 5, wherein the perforation pattern comprises a plurality of perforations between adjacent groups of the plurality of allergen complex cavities.

8. The multi-layer contact patch of claim 1, wherein the allergen complex layer further comprises a scoring pattern.

9. The multi-layer contact patch of claim 1, wherein the plurality of allergen complex cavities comprise a shape pattern comprising at least two distinct shapes, wherein each of the plurality of allergen complex cavities comprise a selected one of the at least two distinct shapes.

10. The multi-layer contact patch of claim 1, further comprising at least one orientation feature, each of the at least one orientation features comprising an opening extending through at least one of the adhesive layer or the allergen complex layer.

11. The multi-layer contact patch of claim 1, further comprising at least one alignment feature, each of the at least one alignment features comprising an opening extending through at least one of the adhesive layer or the allergen complex layer.

12. The multi-layer contact patch of claim 1, wherein each one of the allergen complexes comprises a distinct allergen composition.

13. The multi-layer contact patch of claim 12, wherein the distinct allergen compositions of the allergen complexes are selected to form an allergen panel.

14. The multi-layer contact patch of claim 13, wherein the allergen panel comprises a selected group of allergens according to an expected population incidence rate.

15. The multi-layer contact patch of claim 13, wherein the allergen panel comprises a selected group of allergens according to an exposure profile.

16. The multi-layer contact patch of claim 15, wherein the exposure profile comprises an industry specific exposure profile.

17. The multi-layer contact patch of claim 13, wherein the allergen panel comprises at least one of a mining panel, an agriculture panel, a metal panel, a pediatric panel, a fragrance panel, an oil and gas panel, a refining panel, a manufacturing panel, a carpentry panel, a health care panel, a hospital panel, a dye panel, a textile panel, surgical panel, a dental panel, a hairdresser panel, a specified exposure panel, or a food panel.

18. The multi-layer contact patch of claim 1, wherein at least one of the allergen complexes comprises a control composition.

19. The multi-layer contact patch of claim 1, wherein the absorbent matrix is a gel.

20. The multi-layer contact patch of claim 1, wherein the adhesive layer comprises a surface area that is in direct contact with a user's skin for a duration of a topical allergen test.

21. The multi-layer contact patch of claim 20, wherein the duration is at least two days.

22. The multi-layer contact patch of claim 20, wherein the duration is at least two hours.
